(19) United States
(12) Patent Application Publication
(10) Pub. No.: US 2025/0017964 A1
(43) Pub. Date: Jan. 16, 2025

(54) ENGINEERING OR INDUCING PDGF AND/OR PDGFR SIGNALING TO ENHANCE NK CELL THERAPY

(71) Applicant: City of Hope, Duarte, CA (US)

(72) Inventors: Jianhua Yu, Duarte, CA (US); Michael A. Caligiuri, Duarte, CA (US)

(21) Appl. No.: 18/710,145

(22) PCT Filed: Nov. 14, 2022
(86) PCT No.: PCT/US2022/049851

§ 371 Date: May 14, 2024

Related U.S. Application Data
(60) Provisional application No. 63/279,656, filed on Nov. 15, 2021.

(51) Int. Cl. A61K 35/17; A61K 39/00; A61K 39/39; C07K 14/49; C07K 14/54; C07K 14/71; C12N 5/0783

(52) U.S. Cl. CPC A61K 35/17; A61K 39/39; A61K 39/4613; C07K 14/49; C07K 14/5443; C07K 14/71; C12N 5/0646; C12N 2510/00

(57) ABSTRACT

Provided herein, inter alia, are genetically-modified natural killer (NK) cells capable of expressing platelet-derived growth factor (PDGF) and/or platelet-derived growth factor receptor (PDGFR); pharmaceutical compositions comprising the genetically-modified natural killer (NK) cells; methods of treating cancer with the genetically-modified natural killer (NK) cells; and methods of expanding a population of NK cells using PDGF.

### CROSS-REFERENCE TO RELATED APPLICATIONS

This application claims the benefit of priority to U.S. Application No. 63/279,656 filed Nov. 15, 2021, the disclosure of which is incorporated by reference herein in its entirety.

### STATEMENT AS TO RIGHTS TO INVENTIONS MADE UNDER FEDERALLY SPONSORED RESEARCH AND DEVELOPMENT

This invention was made with government support under AI129582 and CA247550 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file X, created X, with X bytes, machine format IBM-PC, MS Windows operating system, is hereby incorporated by reference.

### BACKGROUND

NK cells, a distinct lymphocyte subset in the circulation, play critical roles in anti-viral and anti-tumor immunity (1). One advantage of NK cells is that they recognize ‘non-self” cells without being activated by specific antigens, allowing a more rapid response than with T cells. This broad cytotoxicity and rapid killing make NK cells ideal for cancer immunotherapy (2). Of note, chimeric-antigen-receptor (CAR)-NK cells have several therapeutic advantages over CAR-T cells (2-4). However, NK cells' shorter lifespan may limit their clinical efficacy. A better understanding of the mechanisms that regulate NK cell survival might therefore improve their clinical application for cancer immunotherapy. The disclosure is directed to this, as well as other, important ends.

### BRIEF SUMMARY

Provided herein is a genetically-modified NK cell that expresses: (i) platelet-derived growth factor D (PDGF-D). In embodiments, the genetically-modified NK cell expresses an increased level of PDGF-D relative to a non-genetically-modified NK cell. Provided herein is a genetically-modified NK cell comprising an exogenous nucleic acid that expresses PDGF-D. In embodiments, the genetically-modified NK cell comprises an exogenous nucleic acid that expresses an increased level of PDGF-D relative to a non-genetically-modified NK cell. Provided herein are methods of treating cancer by administering to a subject in need thereof an effective amount of the genetically-modified NK cells described herein.

Provided herein is a genetically-modified NK cell that expresses platelet-derived growth factor receptor beta (PDGFRβ). In embodiments, the genetically-modified NK cell expresses an increased level of PDGFRβ relative to a non-genetically-modified NK cell. Provided herein is a genetically-modified NK cell comprising an exogenous nucleic acid that expresses PDGFRβ. In embodiments, the genetically-modified NK cell comprises an exogenous nucleic acid that expresses an increased level of PDGFRβ relative to a non-genetically-modified NK cell. Provided herein are methods of treating cancer by administering to a subject in need thereof an effective amount of the genetically-modified NK cells described herein.

Provided herein is a genetically-modified NK cell that expresses PDGF-D and PDGFRβ. In embodiments, the genetically-modified NK cell expresses an increased level of PDGF-D relative to a non-genetically-modified NK cell and an increased level of PDGFRβ relative to a non-genetically-modified NK cell. Provided herein is a genetically-modified NK cell comprising an exogenous nucleic acid that expresses PDGF-D and an exogenous nucleic acid that expresses PDGFRβ. In embodiments, the genetically-modified NK cell comprises an exogenous nucleic acid that expresses an increased level of PDGF-D relative to a non-genetically-modified NK cell and an exogenous nucleic acid that expresses an increased level of PDGFRβ relative to a non-genetically-modified NK cell. Provided herein are methods of treating cancer by administering to a subject in need thereof an effective amount of the genetically-modified NK cells described herein.

These and other embodiments are described in detail herein.

### BRIEF DESCRIPTION OF THE DRAWINGS

FIGS. 1A-1J. IL-15 induces PDGFRβ expression in human NK cells. (A-C) NK cells were purified from PBMCs of healthy donors and stimulated with IL-15 (10 ng/ml) for 24 h. Expression levels of PDGFRα and PDGFRβ were examined by flow cytometry. Data shown are representative dot plots (A), percentages (B), and mean fluorescence intensity (MFI) of PDGFRβ in NK cells (C) (n=20). Resting NK cells were used as the control. (D-E) NK cells were treated with various doses of IL-15 for 24 h (D) or with the same dose of IL-15 (10 ng/ml) at the indicated times (E). Data shown are the percentages of PDGFRβ⁺ NK cells among total NK cells (n=5). (F) Immunofluorescence analysis of PDGFRβ expression on resting and IL-15-treated NK cells. Sodium-potassium ATPase was used as a cell membrane marker. Bar=20 μm. (G) Expression of PDGFRβ in the cytoplasmic, nuclear, and cell membrane fractions of NK cells determined by immunoblotting. (H-J) CD56thin and CD56^{high} NK cells were sorted from PBMCs of healthy donors and treated with IL-15 (10 ng/ml) for 24 h. Expression levels of PDGFRβ were then determined by flow cytometry. Data shown are representative dot plots (H), percentages (I), and MFI (J) of PDGFRβ on NK cells (n=6). Data represent three independent experiments. Data shown are means SD. NS, not significant. *P<0.05 and ****P<0.0001.

FIGS. 2A-2I. IL-15-induced PDGFRβ expression is mediated by PI3K/AKT signaling. (A-B) Primary NK cells were treated with IL-15 (10 ng/ml) for the indicated times. mRNA levels of PDGFRB at different time points (A) (n=3) or at 1 h (B) (n=10) were examined by qPCR. (C-D) Primary NK cells were pretreated with actinomycin D (ActD, 5., g/ml) (C) or cycloheximide (CHX, 20 μg/ml) (D) for 1 h, washed twice with RPMI-1640, and then treated with IL-15 (10 ng/ml) for 24 h. DMSO was used as a control. Expression levels of PDGFRβ were examined by flow cytometry (n=5). Data shown are representative histograms and percentage of inhibition with the equation: % inhibition=100×[1−(DMSO-inhibitor)/DMSO]. (E) Primary NK cells were pretreated with wortmannin (1 μM), afuresertib (10 μM), TPCA-1 (1 μM), rapamycin (10 μM), torin1 (10 μM), decernotinib (10 μM), C118-9 (10 μM), STAT5-IN-1 (10 μM), AZD6244 (10 μM), or CI-1040 (10 μM) for 1 h, washed twice with RPMI 1640, and then treated with IL-15 (10 ng/ml) for 24 h. DMSO was used as control. Data shown are % of inhibition (n=5). The mean value of the inhibitory rate is shown. (F) Luciferase reporter assay shows that p65 activates PDGFRB gene transcription. (G-I) Binding of p65 to the PDGFRB promoter in IL-15-treated NK cells (G) or resting NK cells (H) as determined by ChIP-qPCR or PCR (I) (n=3). Data represent three independent experiments. Data shown are means SD. NS, not significant. *P<0.05, **P<0.01, ***P<0.001, and ****P<0.0001.

FIGS. 3A-3K. PDGF-D enhances NK cell effector functions through NKp44 but not PDGFRβ. (A-E) Primary NK cells were treated with IL-15 (10 ng/ml) in the presence of PDGF-B (50 ng/ml) or PDGF-D (50 ng/ml) for 48 h. Expression levels of IFN-γ, TNF-α, granzyme B, perforin, and CD107a were examined by flow cytometry (n=5). (F-K) Primary NK cells were treated with IL-15 (10 ng/ml) plus PDGF-D (50 ng/ml) in the presence of anti-NKp44 (10 βg/ml) or anti-PDGFRβ (10 μg/ml) for 48 h. Expression levels of IFN-γ, TNF-α, and CD107a were examined by flow cytometry (n=3). Data represent three independent experiments. Data shown are means±SD. NS, not significant. *P<0.05, **P<0.01, and ***P<0.001.

FIGS. 4A-4J. PDGFRβ promotes IL-15-mediated NK cell survival in vitro and in vivo. (A) 5×10⁵ sorted PDGFRβ⁺ (Pos) and PDGFRβ⁻ (Neg) NK cells were cultured in vitro for 7 days in the presence of IL-15 (10 ng/ml). The cells were counted via trypan blue exclusion assay on days 3, 5, and 7 (n=5). (B and C) On day 7, Ki67 levels in PDGFRβ⁺ and PDGFRβ⁻ NK cells were determined by flow cytometry (n=5). (D and E) Annexin V levels in PDGFRβ⁺ and PDGFRβ⁻ NK cells on day 7 were determined by flow cytometry (n=5). (H-J) 1×10⁷ sorted PDGFRβ⁺ or PDGFRβ⁻ IL-15-transduced NK cells were injected into NOD/SCID/IL-2rg (NSG) mice. Blood samples were collected for analysis at the indicated time after adoptive transfer. Data shown are representative dot plots on day 9 after adoptive transfer (H), percentages (I), and absolute numbers (J) of PDGFRβ⁺ and PDGFRβ⁻ NK cells on days 3, 6, and 9 after adoptive transfer (n=5). Data represent three independent experiments. Data shown are means SD. *P<0.05, **P<0.01, ***P<0.001, and ****P<0.0001.

FIGS. 5A-5J. IL-15 induces PDGF-D expression in an autocrine manner. (A) mRNA levels of PDGFA, PDGFB, PDGFC, and PDGF-D in T cells, B cells, and NK cells were analyzed using the BioGPS online tool. (B) mRNA levels of PDGFA, PDGFB, PDGFC, and PDGF-D in T cells, B cells, and NK cells were examined by qPCR (n=10). (C) Primary NK cells were treated with IL-15 (10 ng/ml) for the indicated times. mRNA levels of PDGF-D were examined by qPCR (n=3). (D-E) Representative dot plots and percentages of PDGF-D levels in NK cells after IL-15 (50 ng/ml) treatment for 24 h. Resting NK cells were used as control (n=5). (F) Immunoblotting shows the full length and cleavage of PDGF-D in resting and IL-15-treated NK cells. (G) ELISA assay shows PDGF-D levels in supernatants of NK cell cultures (n=6). (H) Luciferase reporter assay shows that p65 activates PDGF-D gene transcription. (I-J) Binding of p65 to the PDGF-D promoter in IL-15 treated (I) or resting NK cells (J) as determined by ChIP-qPCR (n=3). Data represent three independent experiments. Data shown are means±SD. NS, not significant, *P<0.05, **P<0.01, ***P<0.001, and ****P<0.0001.

FIGS. 6A-6M. IL-15 maintains NK cell survival through a PDGF-D-PDGFRβ autocrine pathway. (A) 1×10⁵ primary NK cells were cultured in vitro for 7 days in the presence of IL-15 (10 ng/ml) and PDGF-D (50 ng/ml). Cells were counted on days 3, 5, and 7 (n=3). (B) 1×10⁵ primary NK cells were cultured in vitro for 7 days in the presence of IL-15 (10 ng/ml) and anti-PDGF-D (10 βg/ml) or control IgG (10 μg/ml). Cells were counted on days 3, 5, and 7 (n=3). (C) 1×10⁵ PDGFRβ-transduced NK cells were cultured in vitro for 7 days in the presence of IL-2 (50 U/ml) and PDGF-D (50 ng/ml). The cells were counted by trypan exclusion assay on days 3, 5, and 7 (n=3). (D) 1×10⁵ primary NK cells were cultured in vitro for 7 days in the presence of IL-15 (10 ng/ml) and PDGF-D (50 ng/ml) as well as anti-PDGFRβ (10 μg/ml). Cells were counted by trypan exclusion assay on days 3, 5, and 7 (n=3). (E) 1×10⁵ primary NK cells were cultured in vitro for 7 days in the presence of IL-15 (10 ng/ml) and PDGF-D (50 ng/ml) as well as anti-NKp44 (10 μg/ml). Cells were counted by trypan exclusion assay on days 3, 5, and 7 (n=3). (F-I) Primary NK cells were cultured in the presence of IL-15 (10 ng/ml) and PDGF-D (50 ng/ml) as well as anti-NKp44 (10 μg/ml) or anti-PDGFRβ (10 Kg/ml) for 48 h. The cultured NK cells were then harvested for immunoblotting to determine protein levels of BCL-2, BCL-XL, and MCL-1 (n=3). (J and K) 1×10⁶ sorted PDGFRβ⁺ (Pos) and PDGFRβ⁻ (Neg) NK cells pretreated with IL-15 (10 ng/ml) for 24 h were cultured in the presence of PDGF-D (50 ng/ml) for 48 h without IL-15. Cell apoptosis and proliferation were analyzed by annexin V staining (J) and Ki-67 staining (K), respectively. Data shown are representative histograms and summary data (n=3). (L and M) 5×10⁶ sorted PDGFRβ⁺ (Pos) and PDGFRβ⁻ (Neg) NK cells overexpressing IL-15 were injected into NOD/SCID/IL-2rg (NSG) mice. The mice were injected intravenously with PDGF-D (1 μg per mouse) or phosphate-buffered saline daily for 3 days. After they were sacrificed, adoptively transferred NK cells (CD45⁺CD56⁺) in their peripheral blood were identified by flow cytometry. The cells were stained for annexin V (L) and Ki-67 (M). Data shown are representative histograms and summary data (n=4). Data represent three independent experiments. Data shown are means±SD. NS, not significant, *P<0.05, **P<0.01, ***P<0.001, and ****P<0.0001.

FIGS. 7A-7F. PDGFRα expression on human and mouse NK cells and the dose and time course effect of IL-2 on the expression of PDGFRβ. (A) Purified human NK cells were stimulated with IL-2 (10 ng/ml), IL-12 (10 ng/ml), IL-15 (10 ng/ml), IL-18 (10 ng/ml), or their combinations for 24 h, followed by determining the expression levels of PDGFRα by flow cytometry. Data shown are representative histograms and mean fluorescence intensity (MFI) of PDGFRα. (B) Purified human NK cells were stimulated with IL-2 (10 ng/ml), IL-12 (10 ng/ml), IL-15 (10 ng/ml), IL-18 (10 ng/ml), or their combinations for 24 h. Expression levels of PDGFRβ were examined by flow cytometry (n=6). (C and D) Purified human NK cells were treated with various doses of IL-2 as indicated for 24 h (C) or with a specific dose of IL-2 (10 ng/ml) for 72 h, as indicated (D). Data shown are the percentages of PDGFRβ⁺ NK cells among total NK cells (n=5). (E) Murine NK cells were purified from the spleen of C57BL/6 mice and stimulated with mouse IL-2 (10 ng/ml), IL-12 (10 ng/ml), IL-15 (10 ng/ml), or IL-12 plus IL-15 for 24 h; then expression levels of PDGFRβ were examined by flow cytometry. Data shown are representative histograms with MFI of PDGFRβ expression. (F) Expression levels of PDGFRβ on NK cells from IL-15 transgenic (Tg) mice were determined by flow cytometry. Data shown are representative histograms. Data represent three independent experiments. Data shown are means SD. ***P<0.001, ****P<0.0001.

FIGS. 8A-8E. Three downstream signaling pathways activated by IL-15 and the effects of their inhibition on IL-15-induced PDGFRβ expression as well as phosphor-p65 levels in NK cells treated with IL-2 or IL-15. (A) Scheme for the three IL-15 signaling pathways and their inhibitors. (B) Representative dot plots of PDGFRβ expression in NK cells treated with specific inhibitors. (C) Binding sites for p65 in the promoter regions of PDGFRB and PDGF-D genes (predicted from http://jaspar.genereg.net). (D and E) Primary human NK cells were treated with IL-2 (10 ng/ml) or IL-15 (10 ng/ml) for 0, 5, 15, 30, 60, or 120 min. The cells were collected for an immunoblot using anti-phosphor (p)-p65, anti-p65, and anti-actin antibodies. The ratios of p-p65 to actin were calculated using ImageJ (n=3). Data shown represent the mean±SD. NS, not significant, **P<0.01, ****P<0.0001.

FIG. 9. ATAC-seq and H3K27ac ChIP-seq data in NK cells. (A) Distribution of ATAC-seq and H3K27 acetylation (ac) ChIP-seq peaks in the promoter region of the PDGFRB locus of CD56^{bright} and CD56^{dim} NK cells, displayed by Integrative Genomics Viewer. Data from the GSE112813 dataset were used for analysis.

FIGS. 10A-10K. PDGFRβ signaling does not affect NK cell effector functions. (A-E) Representative histograms and summary data of IFN-γ, TNF-α, granzyme B, perforin, and CD107a in PDGFRβ⁺ and PDGFRβ⁻ NK cells. Cells were gated on CD56⁺PDGFRβ⁺ or CD56⁺PDGFRβ⁻ cells. (F) PDGFRβ⁺ and PDGFRβ⁻ NK cells were sorted by flow cytometry and co-cultured with ⁵¹ Cr-labeled K562 cells in a 96-well V-bottom plate at ratios of 5:1, 2.5:1, or 1.25:1 for 4 h at 37° C. in a 5% CO₂ incubator. The supernatant was harvested and analyzed using a microbeta scintillation counter (n=3). (G and H) Scheme for the NK cell transduction and representative dot plots of NK cell transduction with empty vector (EV) or lentivirus expressing PDGFRβ. Cells were gated on CD56⁺ cells. (I) Representative dot plots and summary data of CD107a levels in EV or PDGFRβ-expressing NK cells. Cells were gated on CD56⁺GFP⁺ cells. (J) Representative dot plots and summary data of IFN-γ levels in EV or PDGFRβ-overexpressing NK cells. Cells were gated on CD56⁺GFP⁺ cells. (K) Representative histograms and expression levels of CD25, CD69, NKG2D, NKp30, NKp44, NKG2A, and KLRG1 in the PDGFRβ⁺ and PDGFRβ⁻ NK cells (n=5). Cells were gated on CD56⁺PDGFRβ⁺ or CD56⁺PDGFRβ⁻ cells. Data represent three independent experiments. Data shown are means±SD.

FIGS. 11A-11B. CD122 expression in PDGFRβ⁺ and PDGFRβ⁻ NK cells. (A and B) The immunoblot shows the expression levels of the IL-15 receptor β chain CD122 in sorted PDGFRβ⁺ (Pos) and PDGFRβ⁺ (Neg) NK cells purified by flow cytometry. Data represent two independent experiments. Data shown are means±SD. NS, not significant.

FIGS. 12A-12B. A scheme for the NK cell adoptive transfer assay and the transduction efficiency of IL-15. (A) A scheme for the NK cell adoptive transfer assay. Sorted PDGFRβ⁺ or PDGFRβ⁻IL-15-transduced NK cells were injected into NOD/SCID/IL-2rg (NSG) mice. Blood samples were collected for analysis at indicated times after adoptive transfer. (B) NK cells were transduced with soluble IL-15 (as showing in A), resulting in two subsets of NK cells: PDGFRβ⁺ and PDGFRβ⁻. Transduction efficiency of IL-15 was similar for both subsets. EGFR was a marker for IL-15 expression.

FIG. 13. A working model for how autocrine PDGF-D-PDGFRβ signaling and PDGF-D-NKp44 signaling regulate IL-15-mediated NK cell survival and effector functions, respectively. When NK cells are stimulated with IL-15, PDGFRβ and PDGF-D are upregulated via PI3K/AKT/NF-κB signaling. In tum, PDGF-D binds to PDGFRβ to promote NK cell survival, likely through its classic downstream PI3K/AKT and MAPK signaling pathways. PDGF-D can also stimulate NK cells to secrete IFN-γ, TNF-α, and perforin (effector functions) through interaction with NKp44. The figure was created with BioRender. com.

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value. When used with reference to days or weeks, the term “about” refers to +/−2 days or +/−1 day.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine; guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, mlRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., PDGF, PDGFR, etc.) in other proteins with different numbering systems. For example, by performing a simple sequence alignment with a protein (e.g., PDGF, PDGFR, etc.) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M); Valine (V); (6) Phenylalanine (F), Tyrosine (Y), Tryptophan (W); (7) Serine(S), Threonine (T); and (8) Cysteine (C), Methionine (M) (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see e.g., www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2: 482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the protein is the protein as identified by its NCBI or UniProt sequence reference. In embodiments, the protein is the protein as identified by its NCBI or UniProt sequence reference, homolog or functional fragment thereof

The terms “IFN-γ” and “interferon gamma” are used herein according to its plain and ordinary meaning and refer to a dimerized soluble cytokine that is the only member of the type II class of interferons. It plays a role in innate and adaptive immunity against viral, some bacterial and protozoal infections. IFNγ is an important activator of macrophages and inducer of Class II major histocompatibility complex (MHC) molecule expression. The importance of IFNγ in the immune system stems in part from its ability to inhibit viral replication directly and from its immunostimulatory and immunomodulatory effects. IFNγ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops.

The terms “CD107α,” “CD107-alpha,” “lysosomal-associated membrane protein 1,” “LAMP-1” and “lysosome-associated glycoprotein 1” are used in accordance with their plain ordinary meaning and refer to a glycoprotein from a family of lysosome-associated membrane glycoproteins. CD107α is a type I transmembrane protein which is expressed at high or medium levels in at least 76 different normal tissue cell types. It resides primarily across lysosomal membranes, and functions to provide selectins with carbohydrate ligands. CD107α has also been shown to be a marker of degranulation on lymphocytes such as CD8+ and NK cells.

The terms “IL-12,” “IL12,” and “interleukin-12” are used in accordance with their plain ordinary meaning and refer to an interleukin that is naturally produced by dendritic cells, macrophages, neutrophils, and human B-lymphoblastoid cells in response to antigenic stimulation plays an important role in the activities of natural killer cells and T lymphocytes. IL-12 mediates enhancement of the cytotoxic activity of NK cells and CD8+ cytotoxic T lymphocytes. There may be a link between IL-2 and the signal transduction of IL-12 in NK cells. IL-2 stimulates the expression of two IL-12 receptors, IL-12R-β1 and IL-12R-β2, maintaining the expression of a critical protein involved in IL-12 signaling in NK cells. Enhanced functional response is demonstrated by IFN-γ production and killing of target cells.

The terms “IL-15,” “interleukin-15” and “IL15” are used in accordance with their plain ordinary meaning and refer to a cytokine with structural similarity to interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells.

The terms “soluble IL-15,” “sIL-15,” and “sIL15” refer to an IL-15 protein capable of being secreted by an NK cell. In embodiments, the soluble IL-15 includes an IL-2 amino acid signal sequence to facilitate NK cell secretion and/or increase NK cell secretion of the IL-15 relative to the absence of the IL-2 amino acid signal sequence. In embodiments, the soluble IL-15 includes an IL-2 amino acid signal sequence encoded by a nucleic acid including the nucleotide sequence of SEQ ID NO:1. In embodiments, the soluble IL-15 includes an IL-2 amino acid signal sequence encoded by a nucleic acid that is the nucleotide sequence of SEQ ID NO:1. In embodiments, the soluble IL-15 includes an IL-2 amino acid signal sequence encoded by a nucleic acid that has at least 90%, 95%, 96%, 97%, 98%, 99%, or 100% nucleotide sequence identity across the whole nucleic acid sequence or a portion of the nucleic acid sequence (e.g. a 10, 20, or 30 continuous nucleotide portion) compared to a naturally occurring nucleic acid encoding an IL-2 signaling sequence. In embodiments, the soluble IL-15 includes an IL-2 amino acid signal sequence encoded by a nucleic acid that has at least 90%, 95%, 96%, 97%, 98%, 99%, or 100% nucleotide sequence identity across the whole nucleic acid or a portion of the nucleic acid sequence (e.g. a 10, 20, or 30 continuous nucleotide portion) compared to the nucleic acid sequence of SEQ ID NO:1. In embodiments, the soluble IL-15 includes an IL-15 amino acid signal sequence to facilitate NK cell secretion. In embodiments, the soluble IL-15 includes an IL-15 amino acid protein sequence. In embodiments, the soluble IL-15 includes an IL-15 amino acid protein sequence encoded by a nucleic acid including the nucleotide sequence of SEQ ID NO:2. In embodiments, the soluble IL-15 includes an IL-15 amino acid protein sequence encoded by a nucleic acid that is the nucleotide sequence of SEQ ID NO:2. In embodiments, the soluble IL-15 includes an IL-15 amino acid protein sequence encoded by a nucleic acid that has at least 90%, 95%, 96%, 97%, 98%, 99%, or 100% nucleotide sequence identity across the whole nucleic acid or a portion of the nucleic acid sequence (e.g. a 10, 20, or 30 continuous nucleotide portion) compared to the nucleic acid sequence of SEQ ID NO:2. In embodiments, the soluble IL-15 is encoded by a nucleic acid including the nucleotide sequence of SEQ ID NO:3. In embodiments, the soluble IL-15 is encoded by a nucleic acid that is the nucleotide sequence of SEQ ID NO:3. In embodiments, the soluble IL-15 is encoded by a nucleic acid that has at least 90%, 95%, 96%, 97%, 98%, 99%, or 100% nucleotide sequence identity across the whole nucleic acid or a portion of the nucleic acid sequence (e.g. a 10, 20, or 30 continuous nucleotide portion) compared to the nucleic acid sequence of SEQ ID NO:3.

In embodiments, the soluble IL-15 includes an IL-2 amino acid signal sequence including the amino acid sequence of SEQ ID NO:4. In embodiments, the soluble IL-15 includes an IL-2 amino acid signal sequence that is the amino acid sequence of SEQ ID NO:4. In embodiments, the soluble IL-15 includes an IL-2 amino acid signal sequence that has at least 90%, 95%, 96%, 97%, 98%, 99%, or 100% amino acid sequence identity across the whole peptide or a portion of the peptide sequence (e.g. a 5, 10, or 20 continuous amino acid portion) compared to a naturally occurring IL-2 signaling sequence. In embodiments, the soluble IL-15 includes an IL-2 amino acid signal sequence that has at least 90%, 95%, 96%, 97%, 98%, 99%, or 100% amino acid sequence identity across the whole peptide or a portion of the peptide sequence (e.g. a 5, 10, or 20 continuous amino acid portion) compared to the amino acid sequence of SEQ ID NO:4. In embodiments, the soluble IL-15 includes an IL-15 amino acid protein sequence including the amino acid sequence of SEQ ID NO:5. In embodiments, the soluble IL-15 includes an IL-15 amino acid protein sequence that is the amino acid sequence of SEQ ID NO:5. In embodiments, the soluble IL-15 includes an IL-15 amino acid protein sequence that has at least 90%, 95%, 96%, 97%, 98%, 99%, or 100% amino acid sequence identity across the whole peptide or a portion of the peptide sequence (e.g. a 5, 10, or 20 continuous amino acid portion) compared to a naturally occurring IL-15 protein sequence. In embodiments, the soluble IL-15 includes an IL-15 amino acid protein sequence that has at least 90%, 95%, 96%, 97%, 98%, 99%, or 100% amino acid sequence identity across the whole peptide or a portion of the peptide sequence (e.g. a 5, 10, or 20continuous amino acid portion) compared to the amino acid sequence of SEQ ID NO:5. In embodiments, the soluble IL-15 includes the amino acid sequence of SEQ ID NO:6. In embodiments, the soluble IL-15 is the amino acid sequence of SEQ ID NO:6. In embodiments, the soluble IL-15 includes an amino acid sequence that has at least 90%, 95%, 96%, 97%, 98%, 99%, or 100% amino acid sequence identity across the whole peptide or a portion of the peptide sequence (e.g. a 5, 10, or 20 continuous amino acid portion) compared to the amino acid sequence of SEQ ID NO:6.

The terms “IL-18,” “interleukin-18,” “IL 1 8,” “interferon-gamma inducing factor” are used in accordance with their plain ordinary meaning and refer to a proinflammatory cytokine that belongs to the IL-1 superfamily and is produced by macrophages and other cells. IL-18 works by binding to the interleukin-18 receptor, and together with IL-12, it induces cell-mediated immunity following infection with microbial products like lipopolysaccharide (LPS). After stimulation with IL-18, natural killer (NK) cells and certain T cells release another important cytokine called interferon-γ (IFN-γ) or type II interferon that plays an important role in activating the macrophages or other cells.

A “platelet-derived growth factor” or “PDGF” is one among numerous growth factors that regulate cell growth and division. There are five different isoforms of PDGF that activate cellular response through two different receptors. Known ligands include: PDGF-A, PDGF-B, PDGF-C, PDGF-D, and PDGF-AB (a PDGF-A and PDGF-B heterodimer). The ligands interact with the two tyrosine kinase receptor monomers, PDGFRα and PDGFRβ.

A “platelet-derived growth factor D protein” or “PDGF-D” or “PDGF-D protein” as referred to herein includes any of the recombinant or naturally-occurring forms of platelet-derived growth factor D (PDGF-D) also known as iris-expressed growth factor, spinal cord-derived growth factor B, or variants or homologs thereof that maintain PDGF-D activity (e.g. within at least 50%, 80%, 90%, 95%, 96%, 97%, 98%, 99% or 100% activity compared to PDGF-D). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PDGF-D protein. In embodiments, the PDGF-D protein is substantially identical to the protein identified by the UniProt reference number Q9GZP0 or a variant or homolog having substantial identity thereto. In embodiments, the PDGF-D protein has at least 85% sequence identity to the amino acid sequence of SEQ ID NO:7. In embodiments, the PDGF-D protein has at least 90% sequence identity to the amino acid sequence of SEQ ID NO:7. In embodiments, the PDGF-D protein has at least 95% sequence identity to the amino acid sequence of SEQ ID NO:7. In embodiments, the PDGF-D protein comprises the amino acid sequence of SEQ ID NO:7.

A “platelet-derived growth factor receptor” or “PDGFR” is a cell surface tyrosine kinase receptor for members of the platelet-derived growth factor (PDGF) family. The PDGF family consists of PDGF-A, -B, -C and -D. The PDGFs bind to the protein tyrosine kinase receptors PDGF receptor-α and β. The extracellular region of the receptor consists of five immunoglobulin-like domains while the intracellular part is a tyrosine kinase domain. The ligand-binding sites of the receptors are located to the three first immunoglobulin-like domains.

A “platelet-derived growth factor receptor beta protein” or “PDGFR-β” or “PDGFR-β protein” as referred to herein includes any of the recombinant or naturally-occurring forms of platelet-derived growth factor receptor β (PDGFR-β) also known as cluster of beta platelet-derived growth factor receptor, CD140 antigen-like family member B, CD140b, or variants or homologs thereof that maintain PDGFR-β activity (e.g. within at least 50%, 80%, 90%, 95%, 96%, 97%, 98%, 99% or 100% activity compared to PDGFR-(3). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PDGFR-β protein.

In embodiments, the PDGFR-β protein is substantially identical to the protein identified by the UniProt reference number P09619 or a variant or homolog having substantial identity thereto. In embodiments, the PDGF-D protein has at least 85% sequence identity to the amino acid sequence of SEQ ID NO:8. In embodiments, the PDGF-D protein has at least 90% sequence identity to the amino acid sequence of SEQ ID NO:8. In embodiments, the PDGF-D protein has at least 95% sequence identity to the amino acid sequence of SEQ ID NO:8. In embodiments, the PDGF-D protein comprises the amino acid sequence of SEQ ID NO:8.

In embodiments, the PDGFR-β protein is substantially identical to the protein identified by the UniProt reference number E5J14 or a variant or homolog having substantial identity thereto. In embodiments, the PDGF-D protein has at least 85% sequence identity to the amino acid sequence of SEQ ID NO:9. In embodiments, the PDGF-D protein has at least 90% sequence identity to the amino acid sequence of SEQ ID NO:9. In embodiments, the PDGF-D protein has at least 95% sequence identity to the amino acid sequence of SEQ ID NO:9. In embodiments, the PDGF-D protein comprises the amino acid sequence of SEQ ID NO:9.

In embodiments, the PDGFR-β protein is substantially identical to the protein identified by the UniProt reference number E5RII0 or a variant or homolog having substantial identity thereto. In embodiments, the PDGF-D protein has at least 85% sequence identity to the amino acid sequence of SEQ ID NO:10. In embodiments, the PDGF-D protein has at least 90% sequence identity to the amino acid sequence of SEQ ID NO:10. In embodiments, the PDGF-D protein has at least 95% sequence identity to the amino acid sequence of SEQ ID NO:10. In embodiments, the PDGF-D protein comprises the amino acid sequence of SEQ ID NO:10.

“Truncated epidermal growth factor” or “truncated EGFR” or “tEGFR” refers to epidermal growth factor that is devoid of intracellular receptor tyrosine kinase activity. In embodiments, tEGFR is devoid of extracellular N-terminal ligand binding domains and intracellular receptor tyrosine kinase activity. In embodiments, tEGFR is devoid of extracellular N-terminal ligand binding domains and intracellular receptor tyrosine kinase activity, but retains the native amino acid sequence, type I transmembrane cell surface localization, and a conformationally intact binding epitope. In embodiments, tEGFR comprises EGFR Domain III, the EGFR transmembrane domain, and the EGFR Domain IV. In embodiments, tEGFR has at least 85% sequence identity to the protein identified by UniProt reference number Q9H3C8. In embodiments, tEGFR has at least 90% sequence identity to the protein identified by UniProt reference number Q9H3C8. In embodiments, tEGFR has at least 95% sequence identity to the protein identified by UniProt reference number Q9H3C8. In embodiments, tEGFR has the sequence set for the protein identified by UniProt reference number Q9H3C8.

A “PD-1 protein” or “PD-1” as referred to herein includes any of the recombinant or naturally-occurring forms of the Programmed cell death protein 1 (PD-1) also known as cluster of differentiation 279 (CD 279) or variants or homologs thereof that maintain PD-1protein activity (e.g. within at least 50%, 80%, 90%, 95%, 96%, 97%, 98%, 99% or 100% activity compared to PD-1 protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-1 protein. In embodiments, the PD-1 protein is substantially identical to the protein identified by the UniProt reference number Q15116 or a variant or homolog having substantial identity thereto. In embodiments, the PD-1 protein is substantially identical to the protein identified by the UniProt reference number Q02242 or a variant or homolog having substantial identity thereto.

A “PD-L1 ” or “PD-L1 protein” as referred to herein includes any of the recombinant or naturally-occurring forms of programmed death ligand 1 (PD-L1) also known as cluster of differentiation 274 (CD 274) or variants or homologs thereof that maintain PD-L1 activity (e.g. within at least 50%, 80%, 90%, 95%, 96%, 97%, 98%, 99% or 100% activity compared to PD-L1). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-L1 protein. In embodiments, the PD-L1 protein is substantially identical to the protein identified by the UniProt reference number Q9NZQ7 or a variant or homolog having substantial identity thereto.

The terms “genetically-modified” and “recombinant” when used with reference, e.g., to a cell, nucleic acid, protein, or vector, indicates that the cell, nucleic acid, protein or vector, has been modified by the introduction of an exogenous nucleic acid or protein or the alteration of a native nucleic acid or protein, or that the cell is derived from a cell so modified. Thus, for example, genetically-modified cells express genes that are not found within the native (non-recombinant) form of the cell or express native genes that are otherwise abnormally expressed, under expressed, or not expressed at all. Transgenic cells and plants are those that express a heterologous gene or coding sequence, typically as a result of recombinant methods.

The term “non-genetically-modified NK cell” refers to a NK cell that has not been genetically-modified. In embodiments, a non-genetically-modified NK cell is a NK cell that has not been genetically modified to express PDGF and/or PDGFR.

The terms “exogenous” and “heterologous” refer to a molecule or substance (e.g., a compound, nucleic acid, or protein) in a host organism (e.g., NK cell) that does not naturally have that molecule or substance (e.g., a compound, nucleic acid or protein). For example, an “exogenous nucleic acid” as referred to herein is a nucleic acid that is not naturally occurring in in a host organism (e.g., NK cell). For example, an exogenous nucleic acid may be produced by transforming a cell with a plasmid including that nucleic acid. Conversely, the term “endogenous nucleic acid” refers to a molecule or substance that is naturally occurring within a given host organism (e.g., NK cell).

The term “expression” or “expresses” is used in accordance with its plain ordinary meaning and refers to any step involved in the production of a protein including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry).

The term “constitutive expression” or “constitutively expresses” refers to a gene that is transcribed in an ongoing manner, e.g., the gene is constantly being transcribed at a constant level.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid,” “vector” or “expression vector” refer to a nucleic acid molecule that encodes for genes and/or regulatory elements necessary for the expression of genes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well. In embodiments, the expression vector comprises an inducible promoter. In embodiments, the inducible promoter is a hypoxia-inducible promoter.

The term “promoter” refers to a DNA sequence located near and upstream to the transcription initiation site of the gene recognized by the synthetic machinery of the cell, or introduced synthetic machinery, required to initiate the specific transcription of a gene. In embodiments, the promoter is an inducible promoter. In embodiments, the promoter is a constitutive promoter. Inducible promoters are regulated promoters that become active in the cell only in response to a specific stimulus. Constitutive promoters are unregulated promoters that are active in all circumstances in the cell.

The term “inducible control” refers to a gene or nucleic acid sequence that has transcription controlled by an inducible promoter.

The terms “transfection,” “transduction,” “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect and human cells.

The terms “natural killer cell” and “NK cell” are used in accordance with their plain ordinary meaning and refer to a type of cytotoxic lymphocyte involved in the innate immune system. The role NK cells play is typically analogous to that of cytotoxic T cells in the vertebrate adaptive immune response. NK cells may provide rapid responses to virus-infected cells, acting at around 3 days after infection, and respond to tumor formation. Typically, immune cells detect major histocompatibility complex (MHC) presented on infected cell surfaces, triggering cytokine release, causing lysis or apoptosis. NK cells typically have the ability to recognize stressed cells in the absence of antibodies and MHC, allowing for a much faster immune reaction.

The term “COH06” or “enhanced CB-NK cells” or “PD-L1(+) natural killer cells” or “population of PD-L1(+) natural killer cells” refers to activated PD-L1(+) cord blood (CB) NK cells successfully transduced with a retrovirus encoding the sIL-15 gene and the tEGFR gene.

The product COH06 is defined in Table 8.1.2.1 (Release Testing of COH06). Following treatment with the retrovirus, RRV-sIL-15 JEGFR, the cells are stimulated with cytokines IL-12 and IL-18 and following expansion, cryopreserved in CryoStor® CS5 (STEMCELL™) using a controlled rate freezer. The product administered to patients also contains PD-L1(+) CB-NK cells not transduced with sIL-15 or tEGFR. These cells are referred to as activated PD-L1 (+) untransduced NK cells. Manufacture of the investigational NK cell product will be conducted under cGMP at the Center for Biomedicine and Genetics (CBG) located at the City of Hope. Cord blood donors are screened in accordance with 21 CFR 1270. The NK cells are purified from cord blood using a RosetteSep Human NK Cell Enrichment Cocktail followed by centrifugation through a Ficoll-Paque gradient to develop an umbilical cord NK cell bank. The enriched NK cells are cryopreserved in CryoStor CS5; one million cells are frozen separately to be used in the assessment of the cells' proliferation capacity (17 days expansion at small scale in the presence of IL-2 and irradiated K562 feeders). NK cells that have demonstrated sufficient expansion capacity from the 1×10⁶ aliquot such that the remaining cells would be capable of generating at least 2×10⁹ cells post full-scale expansion are subsequently thawed and will be used for cell therapy productions. The NK cells will then be co-cultured with the irradiated K562 feeder cells expressing membrane-bound (IL-21) and CD-137L and exogenous IL-2. On day 7, expanded NK cells are transduced with the retroviral vector (RRV_sIL-15tEGFR) carrying the human IL-15 gene and the truncated EGFR. Following transduction, the cells are further expanded with additional irradiated K562 feeder cells. On day 16, the cytokines IL-18 and IL-12 are added to the cell culture to harvest to upregulate endogenous expression of PD-L1 on the sIL15+ NK cells. On day 17, the cells are harvested and cryopreserved. The Release Testing for product COH06 is shown in Table 1. PD-L1(+) natural killer cells are described in WO 2020/264043, the disclosure of which is incorporated by reference herein in its entirety.

TABLE 1

| Assay | Testing Lab | Specification |
|---|---|---|
| Sterility USP | BioReliance | No Growth |
| Mycolasma USP | BioReliance | Negative |
| Endotoxin | BioReliance | <10 EU/mL |
| Purity | CBG COH QC | CD56(+) ≥80%; |
| | | CD3(+) ≤10% |
| Viability at Thaw | CBG COH QC | ≥70% |
| Viable Cell Concentration | CBG COH QC | Report Result |
| at Thaw | | |
| Replication Competent | Indiana | Not Detected |
| Retrovirus | University | |
| (for RD114 pseudotyped) | | |
| VCN Assay | COH APCF Lab | ≤5.0 Copies Per Cell |
| PDL1 Expression | CBG COH QC | ≥10% |
| (FACS Assay) | | |
| tEGFR Expression | CBG COH QC | ≥10% |
| (FACS Assay) | | |
| CD137/IL21 Feeder | CBG COH QC | Residual K562 |
| Cell Residual Assay | | Feeder Cells <10% |
| (FACS Assay) | | |
| IL-15 ELISA | CBG COH QC | Detectable IL-15, |
| | | >1 pg/ml |

The term “PD-L1(+) natural killer (NK) cells” are natural killer cells that express PD-L1 protein. In embodiments, the PD-L1(+) natural killer cell expresses cell surface PD-L1. In embodiments, the PD-L1(+) natural killer cell is a recombinant PD-L1(+) natural killer cell.

The term “population of PD-L1(+) natural killer (NK) cells” refers to a plurality of PD-L1(+) natural killer (NK) cells.

The term “T cells” or “T lymphocytes” are used in accordance with their plain ordinary meaning and refer to a type of lymphocyte (a subtype of white blood cell) involved in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

The term “feeder cell” or “feeders” are used in accordance with their plain ordinary meaning and refer to adherent growth-arrested, but viable and bioactive, cells. These cells may be used as a substratum to condition the medium on which other cells, particularly at low or clonal density, are grown. In embodiments, the cells of the feeder layer are irradiated or otherwise treated so that they will not proliferate.

The terms “K562 cell” and “K562 cell line” are used in accordance with their plain ordinary meaning and refer to a human immortalized myelogenous leukemia cell line derived from a 53-year-old female chronic myelogenous leukemia patient in blast crisis. K562 cells are of the erythroleukemia type. The cells are non-adherent and rounded, are positive for the bcr:abl fusion gene, and bear some proteomic resemblance to both undifferentiated granulocytes and erythrocytes.

The term “chimeric antigen receptor” or “CAR” refers to a chimeric polypeptide which comprises a polypeptide sequence that recognizes a target antigen (an antigen-recognition domain) linked to a transmembrane polypeptide and intracellular domain polypeptide selected to activate the T cell and provide specific immunity. The antigen-recognition domain may be a single-chain variable fragment (ScFv), or may, for example, be derived from other molecules such as, for example, a T cell receptor or Pattern Recognition Receptor. The intracellular domain comprises at least one polypeptide which causes activation of the T cell, such as, for example, but not limited to, CD3 zeta, and, for example, co-stimulatory molecules, for example, but not limited to, CD28, OX40 and 4-1 BB. The term “chimeric antigen receptor” may also refer to chimeric receptors that are not derived from antibodies, but are chimeric T cell receptors. These chimeric T cell receptors may comprise a polypeptide sequence that recognizes a target antigen, where the recognition sequence may be, for example, but not limited to, the recognition sequence derived from a T cell receptor or an scFv. The intracellular domain polypeptides are those that act to activate the T cell.

The terms “control” or “control experiment” are used in accordance with its plain ordinary meaning and refer to an experiment in which the subjects or reagents of the experiment are treated as in a parallel experiment except for omission of a procedure, reagent, or variable of the experiment. In embodiments, the control is used as a standard of comparison in evaluating experimental effects. In embodiments, a control is the measurement of the activity of a protein in the absence of a compound as described herein (including embodiments and examples).

“Contacting” is used in accordance with its plain ordinary meaning and refers to the process of allowing at least two distinct species (e.g., PDGF-D and an NK cell) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The terms “activation,” “activate,” “activating,” “activator” and the like are used in accordance with its plain ordinary meaning and refer to an interaction that positively affects (e.g. increasing) the activity or function of a protein or cell relative to the activity or function of the protein or cell in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein that is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. are used in accordance with its plain ordinary meaning and refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

The terms “inhibition,” “inhibit,” “inhibiting” and the like are used in accordance with its plain ordinary meaning and refer to an interaction that negatively affecting (e.g. decreasing) the activity or function of the protein or cell relative to the activity or function of the protein or cell in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein or cell from an indirect interaction (e.g. an inhibitor binds to a protein that activates the target protein, thereby preventing target protein activation or cell activations).

The terms “inhibitor,” “repressor” or “antagonist” or “downregulator” are used in accordance with its plain ordinary meaning and refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “signaling pathway” is used in accordance with its plain ordinary meaning and refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “cytokine” is used in accordance with its plain ordinary meaning and refers to a broad category of small proteins that are important in cell signaling. Cytokines are peptides, and cannot cross the lipid bilayer of cells to enter the cytoplasm. Cytokines are involved in autocrine signaling, paracrine signaling and endocrine signaling as immunomodulating agents. Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell.

The term “immunotherapy, immunotherapeutic” and “immunotherapeutic agent” are used in accordance with their plain ordinary meaning and refer to the treatment of disease by activating or suppressing the immune system. Immunotherapies designed to elicit or amplify an immune response are classified as activation immunotherapies, while immunotherapies that reduce or suppress are classified as suppression immunotherapies. Such immunotherapeutic agents include antibodies and cell therapy.

The term “checkpoint inhibitor” is used in accordance with its plain ordinary meaning and refers to a drug, often made of antibodies, that unleashes an immune system attack on cancer cells. An important part of the immune system is its ability to tell between normal cells in the body and those it sees as “foreign.” This lets the immune system attack the foreign cells while leaving the normal cells alone. To do this, it uses “checkpoints” which are molecules on certain immune cells that need to be activated (or inactivated) to start an immune response. Cancer cells sometimes find ways to use these checkpoints to avoid being attacked by the immune system. Drugs that target these checkpoints are known as checkpoint inhibitors.

The term “immune response” is used in accordance with its plain ordinary meaning and refers to a response by an organism that protects against disease. The response can be mounted by the innate immune system or by the adaptive immune system, as well known in the art.

The terms “tumor microenvironment,” “TME,” and “cancer microenvironment” are used in accordance with its plain ordinary meaning and refer to the non-neoplastic cellular environment of a tumor, including blood vessels, immune cells, fibroblasts, cytokines, chemokines, non-cancerous cells present in the tumor, and proteins produced.

The terms “cell therapy” and “cellular therapy” are used in accordance with their plain ordinary meaning and refer to therapy in which cellular material such as for example cells is injected, grafted or implanted into a patient. The cells may he living cells. in embodiments, the cells are NK

The term “anticancer agent” and “anticancer therapy” are used in accordance with their plain ordinary meaning and refer to a molecule or composition (e.g. compound, peptide, protein, nucleic acid, drug, antagonist, inhibitor, modulator) or regimen used to treat cancer through destruction or inhibition of cancer cells or tissues. Anticancer therapy includes chemotherapy, radiation therapy, surgery, targeted therapy, immunotherapy, and cell therapy Anticancer agents and/or anticancer therapy may be selective for certain cancers or certain tissues. In embodiments, an anti-cancer therapy is an immunotherapy. In embodiments, anticancer agent or therapy may include a checkpoint inhibitor (e.g. administration of an effective amount of a checkpoint inhibitor). In embodiments, the anti-cancer agent or therapy is a cell therapy.

The term “patient” or “subject in need thereof” is used in accordance with its plain ordinary meaning and refers to a living organism suffering from or prone to a disease or condition that can be treated by administration of a composition, compound, or method as provided herein. Non-limiting examples include humans, other mammals, bovines, rats, mice, dogs, monkeys, goat, sheep, cows, and other non-mammalian animals. In embodiments, a patient is human. In embodiments, the subject has cancer.

“Treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “treating” or “treatment” are used in accordance with its plain ordinary meaning and refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating includes preventing. In embodiments, treating does not include preventing.

The term “prevent” is used in accordance with its plain ordinary meaning and refers to a decrease in the occurrence of disease symptoms in a patient. The prevention may be complete (no detectable symptoms) or partial, such that fewer symptoms are observed than would likely occur absent treatment.

The term “cancer” is used in accordance with its plain ordinary meaning and refers to all types of cancer, neoplasm or malignant tumors found in mammals (e.g. humans), including leukemias, lymphomas, carcinomas and sarcomas. Examples of cancers that may be treated with a compound, composition, or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is lung cancer. In embodiments, the cancer is leukemia.

The term “leukemia” is used in accordance with its plain ordinary meaning and refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Examples of leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia. In embodiments, the cancer is acute myeloid leukemia.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “an effective amount,” “a thereutically effective amount.” “therapeutically effective dose or amount” and the like is intended an amount of cells, agents, or compounds described herein that brings about a positive therapeutic response in a subject in need of, such as an amount that restores function and/or results in the elimination and/or reduction of tumor and/or cancer cells. The exact amount (of cells or agents) required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein. A “combined therapeutically effective amount” or “combined therapeutically effective dose or amount dose” refers a combination of therapies that together brings about a positive therapeutic response in a subject in need of, such as an amount that restores function and/or results in the elimination and/or reduction of tumor and/or cancer cells.

The term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions can also be delivered as nanoparticles.

As used herein, the term “pharmaceutically acceptable” is used synonymously with “physiologically acceptable” and “pharmacologically acceptable.” A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

### Genetically-Modified NK Cells

Provided herein are NK cells that are capable of expressing PDGF and/or PDGFR. It is contemplated that increased expression of PDGF/PDGFR increases NK cell survival compared to an NK cell that does not express PDGF/PDGFR or expresses low levels of PDGF/PDGFR. Applicant has found that contacting an NK cell with IL-15 increases expression of PDGF/PDGFR, thereby enhancing NK cell survival. Thus, the NK cells provided herein including embodiments thereof will have enhanced cell expansion and/or persistence. The NK cells provided herein will further have increased effector function in NK cell-based immunotherapies. Thus, in an aspect is provided a natural killer (NK) cell capable of expressing platelet-derived growth factor (PDGF). In embodiments, the natural killer (NK) cell is capable of recombinantly expressing platelet-derived growth factor (PDGF). In embodiments, the natural killer (NK) cell expresses platelet-derived growth factor (PDGF). In embodiments, the natural killer (NK) cell recombinantly expresses platelet-derived growth factor (PDGF). In an aspect is provided a natural kill (NK) cell capable of expressing an increased level of platelet-derived growth factor (PDGF) relative to a control. In embodiments, the natural kill (NK) cell is capable of recombinantly expressing an increased level of platelet-derived growth factor (PDGF) relative to a control. In embodiments, the natural kill (NK) cell expresses an increased level of platelet-derived growth factor (PDGF) relative to a control. In embodiments, the natural kill (NK) cell recombinantly expresses an increased level of platelet-derived growth factor (PDGF) relative to a control. In embodiments, the PDGF is PDGF-D. In an aspect is provided a NK cell capable of expressing platelet-derived growth factor receptor (PDGFR). In embodiments, the NK cell is capable of recombinantly expressing PDGFR. In embodiments, the NK cell expresses PDGFR. In embodiments, the NK cell recombinantly expresses PDGFR. In another aspect is provided a NK cell capable of expressing an increased level of PDGFR relative to a control. In embodiments, the NK cell is capable of recombinantly expressing an increased level of PDGFR relative to a control. In embodiments, the natural killer (NK) cell expresses an increased level of PDGFR relative to a control. In embodiments, the NK cell recombinantly expresses an increased level of PDGFR relative to a control. In embodiments, the PDGFR is PDGFRβ. In embodiments, the NK cell is capable of expressing platelet-derived growth factor receptor-beta (PDGFRβ). In embodiments, the NK cell is capable of recombinantly expressing PDGFRβ. In embodiments, the NK cell expresses PDGFRβ. In embodiments, the NK cell recombinantly expresses PDGFRβ. In another aspect is provided a NK cell capable of expressing an increased level of PDGFRβ relative to a control. In embodiments, the NK cell is capable of recombinantly expressing an increased level of PDGFRβ relative to a control. In embodiments, the natural killer (NK) cell expresses an increased level of PDGFRβ relative to a control. In embodiments, the NK cell recombinantly expresses an increased level of PDGFRβ relative to a control.

In embodiments, the NK cell provided herein expresses at least 10% higher levels of PDGF/PDGFR than a control. In embodiments, the NK cell provided herein expresses at least 20% higher levels of PDGF/PDGFR than a control. In embodiments, the NK cell provided herein expresses at least 30% higher levels of PDGF/PDGFR than a control. In embodiments, the NK cell provided herein expresses at least 40% higher levels of PDGF/PDGFR than a control. In embodiments, the NK cell provided herein expresses at least 50% higher levels of PDGF/PDGFR than a control. In embodiments, the NK cell provided herein expresses at least 60% higher levels of PDGF/PDGFR than a control. In embodiments, the NK cell provided herein expresses at least 70% higher levels of PDGF/PDGFR than a control. In embodiments, the NK cell provided herein expresses at least 80% higher levels of PDGF/PDGFR than a control. In embodiments, the NK cell provided herein expresses at least 90% higher levels of PDGF/PDGFR than a control.

In embodiments, the NK cell provided herein expresses at least about 10% to about 90% higher levels of PDGF/PDGFR than a control. In embodiments, the NK cell provided herein expresses at least about 20% to about 90% higher levels of PDGF/PDGFR than a control. In embodiments, the NK cell provided herein expresses at least about 30% to about 90% higher levels of PDGF/PDGFR than a control. In embodiments, the NK cell provided herein expresses at least about 40% to about 90% higher levels of PDGF/PDGFR than a control. In embodiments, the NK cell provided herein expresses at least about 50% to about 90% higher levels of PDGF/PDGFR than a control. In embodiments, the NK cell provided herein expresses at least about 60% to about 90% higher levels of PDGF/PDGFR than a control. In embodiments, the NK cell provided herein expresses at least about 70% to about 90% higher levels of PDGF/PDGFR than a control. In embodiments, the NK cell provided herein expresses at least about 80% to about 90% higher levels of PDGF/PDGFR than a control.

In embodiments, the NK cell provided herein expresses at least about 10% to about 80% higher levels of PDGF/PDGFR than a control. In embodiments, the NK cell provided herein expresses at least about 10% to about 70% higher levels of PDGF/PDGFR than a control. In embodiments, the NK cell provided herein expresses at least about 10% to about 60% higher levels of PDGF/PDGFR than a control. In embodiments, the NK cell provided herein expresses at least about 10% to about 50% higher levels of PDGF/PDGFR than a control. In embodiments, the NK cell provided herein expresses at least about 10% to about 40% higher levels of PDGF/PDGFR than a control. In embodiments, the NK cell provided herein expresses at least about 10% to about 30% higher levels of PDGF/PDGFR than a control. In embodiments, the NK cell provided herein expresses at least about 10% to about 20% higher levels of PDGF/PDGFR than a control. In embodiments, the NK cell provided herein expresses at least about 10%, 20%, 30%, 40%, 50%, 60%, 70%, 80%, or 90% higher levels of PDGF/PDGFR than a control.

Provided herein is a genetically-modified NK cell that expresses platelet-derived growth factor (PDGF). In embodiments, the genetically-modified NK cell comprises an exogenous nucleic acid that expresses PDGF. In embodiments, the genetically-modified NK cell expresses an increased level of PDGF relative to a non-genetically-modified NK cell. In embodiments, the genetically-modified NK cell comprises an exogenous nucleic acid that expresses an increased level of PDGF relative to a non-genetically-modified NK cell. In embodiments, the genetically-modified NK cell constitutively expresses PDGF.

Provided herein is a genetically-modified NK cell expresses platelet-derived growth factor D (PDGF-D). In embodiments, the genetically-modified NK cell comprises an exogenous nucleic acid that expresses PDGF-D. In embodiments, the genetically-modified NK cell expresses an increased level of PDGF-D relative to a non-genetically-modified NK cell. In embodiments, the genetically-modified NK cell comprises an exogenous nucleic acid that expresses an increased level of PDGF-D relative to a non-genetically-modified NK cell. In embodiments, the genetically-modified NK cell constitutively expresses PDGF-D.

Provided herein is a genetically-modified NK cell that expresses PDGF-D having at least 85% sequence identity to SEQ ID NO:7. In embodiments, the genetically-modified NK cell expresses PDGF-D having at least 90% sequence identity to SEQ ID NO:7. In embodiments, the genetically-modified NK cell expresses PDGF-D having at least 95% sequence identity to SEQ ID NO:7. In embodiments, the genetically-modified NK cell expresses PDGF-D having SEQ ID NO:7. In embodiments, the genetically-modified NK cell comprises an exogenous nucleic acid that expresses PDGF-D having at least 85% sequence identity to SEQ ID NO:7. In embodiments, the genetically-modified NK cell comprises an exogenous nucleic acid that expresses PDGF-D having at least 90% sequence identity to SEQ ID NO:7. In embodiments, the genetically-modified NK cell comprises an exogenous nucleic acid that expresses PDGF-D having at least 95% sequence identity to SEQ ID NO:7. In embodiments, the genetically-modified NK cell comprises an exogenous nucleic acid that expresses PDGF-D having SEQ ID NO:7. In embodiments, the genetically-modified NK cell expresses an increased level of PDGF-D having at least 85% sequence identity to SEQ ID NO:7 relative to a non-genetically-modified NK cell. In embodiments, the genetically-modified NK cell expresses an increased level of PDGF-D having at least 90% sequence identity to SEQ ID NO:7 relative to a non-genetically-modified NK cell. In embodiments, the genetically-modified NK cell expresses an increased level of PDGF-D having at least 95% sequence identity to SEQ ID NO:7 relative to a non-genetically-modified NK cell. In embodiments, the genetically-modified NK cell expresses an increased level of PDGF-D having SEQ ID NO:7 relative to a non-genetically-modified NK cell. In embodiments, the genetically-modified NK cell comprises an exogenous nucleic acid that expresses an increased level of PDGF-D having at least 85% sequence identity to SEQ ID NO:7 relative to a non-genetically-modified NK cell. In embodiments, the genetically-modified NK cell comprises an exogenous nucleic acid that expresses an increased level of PDGF-D having at least 90% sequence identity to SEQ ID NO:7 relative to a non-genetically-modified NK cell. In embodiments, the genetically-modified NK cell comprises an exogenous nucleic acid that expresses an increased level of PDGF-D having at least 95% sequence identity to SEQ ID NO:7 relative to a non-genetically-modified NK cell. In embodiments, the genetically-modified NK cell comprises an exogenous nucleic acid that expresses an increased level of PDGF-D having SEQ ID NO:7 relative to a non-genetically-modified NK cell. In embodiments, the genetically-modified NK cell constitutively expresses PDGF-D having at least 85% sequence identity to SEQ ID NO:7. In embodiments, the genetically-modified NK cell constitutively expresses PDGF-D having at least 90% sequence identity to SEQ ID NO:7. In embodiments, the genetically-modified NK cell constitutively expresses PDGF-D having at least 95% sequence identity to SEQ ID NO:7. In embodiments, the genetically-modified NK cell constitutively expresses PDGF-D having SEQ ID NO:7.

Provided herein is a genetically-modified NK cell that expresses platelet-derived growth factor receptor (PDGFR). In embodiments, the genetically-modified NK cell comprises an exogenous nucleic acid that expresses PDGFR. In embodiments, the genetically-modified NK cell expresses an increased level of PDGFR relative to a non-genetically-modified NK cell. In embodiments, the genetically-modified NK cell comprises an exogenous nucleic acid that expresses an increased level of PDGFR relative to a non-genetically-modified NK cell. In embodiments, the genetically-modified NK cell constitutively expresses PDGFR.

Provided herein is a genetically-modified NK cell expresses platelet-derived growth factor receptor beta (PDGFRβ). In embodiments, the genetically-modified NK cell comprises an exogenous nucleic acid that expresses PDGFRβ. In embodiments, the genetically-modified NK cell expresses an increased level of PDGFRβ relative to a non-genetically-modified NK cell. In embodiments, the genetically-modified NK cell comprises an exogenous nucleic acid that expresses an increased level of PDGFRβ relative to a non-genetically-modified NK cell. In embodiments, the genetically-modified NK cell constitutively expresses PDGFRβ.

Provided herein is a genetically-modified NK cell that expresses PDGFRβ having at least 85% sequence identity to SEQ ID NO:8. In embodiments, the genetically-modified NK cell expresses PDGFRβ having at least 90% sequence identity to SEQ ID NO:8. In embodiments, the genetically-modified NK cell expresses PDGFRβ having at least 95% sequence identity to SEQ ID NO:8. In embodiments, the genetically-modified NK cell expresses PDGFRβ having SEQ ID NO:8. In embodiments, the genetically-modified NK cell comprises an exogenous nucleic acid that expresses PDGFRβ having at least 85% sequence identity to SEQ ID NO:8. In embodiments, the genetically-modified NK cell comprises an exogenous nucleic acid that expresses PDGFRβ having at least 90% sequence identity to SEQ ID NO:8. In embodiments, the genetically-modified NK cell comprises an exogenous nucleic acid that expresses PDGFRβ having at least 95% sequence identity to SEQ ID NO:8. In embodiments, the genetically-modified NK cell comprises an exogenous nucleic acid that expresses PDGFRβ having SEQ ID NO:8. In embodiments, the genetically-modified NK cell expresses an increased level of PDGFRβ having at least 85% sequence identity to SEQ ID NO:8 relative to a non-genetically-modified NK cell. In embodiments, the genetically-modified NK cell expresses an increased level of PDGFRβ having at least 90% sequence identity to SEQ ID NO:8 relative to a non-genetically-modified NK cell. In embodiments, the genetically-modified NK cell expresses an increased level of PDGFRβ having at least 95% sequence identity to SEQ ID NO:8 relative to a non-genetically-modified NK cell. In embodiments, the genetically-modified NK cell expresses an increased level of PDGFRβhaving SEQ ID NO:8 relative to a non-genetically-modified NK cell. In embodiments, the genetically-modified NK cell comprises an exogenous nucleic acid that expresses an increased level of PDGFRβ having at least 85% sequence identity to SEQ ID NO:8 relative to a non-genetically-modified NK cell. In embodiments, the genetically-modified NK cell comprises an exogenous nucleic acid that expresses an increased level of PDGFRβ having at least 90% sequence identity to SEQ ID NO:8 relative to a non-genetically-modified NK cell. In embodiments, the genetically-modified NK cell comprises an exogenous nucleic acid that expresses an increased level of PDGFRβ having at least 95% sequence identity to SEQ ID NO:8 relative to a non-genetically-modified NK cell. In embodiments, the genetically-modified NK cell comprises an exogenous nucleic acid that expresses an increased level of PDGFRβ having SEQ ID NO:8 relative to a non-genetically-modified NK cell. In embodiments, the genetically-modified NK cell constitutively expresses PDGFRβ having at least 85% sequence identity to SEQ ID NO:8. In embodiments, the genetically-modified NK cell constitutively expresses PDGFRβ having at least 90% sequence identity to SEQ ID NO:8. In embodiments, the genetically-modified NK cell constitutively expresses PDGFRβ having at least 95% sequence identity to SEQ ID NO:8. In embodiments, the genetically-modified NK cell constitutively expresses PDGFRβ having SEQ ID NO:8.

Provided herein is a genetically-modified NK cell that expresses PDGFRβ having at least 85% sequence identity to SEQ ID NO:9. In embodiments, the genetically-modified NK cell expresses PDGFRβ having at least 90% sequence identity to SEQ ID NO:9. In embodiments, the genetically-modified NK cell expresses PDGFRβ having at least 95% sequence identity to SEQ ID NO:9. In embodiments, the genetically-modified NK cell expresses PDGFRβ having SEQ ID NO:9. In embodiments, the genetically-modified NK cell comprises an exogenous nucleic acid that expresses PDGFRβ having at least 85% sequence identity to SEQ ID NO:9. In embodiments, the genetically-modified NK cell comprises an exogenous nucleic acid that expresses PDGFRβ having at least 90% sequence identity to SEQ ID NO:9. In embodiments, the genetically-modified NK cell comprises an exogenous nucleic acid that expresses PDGFRβ having at least 95% sequence identity to SEQ ID NO:9. In embodiments, the genetically-modified NK cell comprises an exogenous nucleic acid that expresses PDGFRβ having SEQ ID NO:9. In embodiments, the genetically-modified NK cell expresses an increased level of PDGFRβ having at least 85% sequence identity to SEQ ID NO:9 relative to a non-genetically-modified NK cell. In embodiments, the genetically-modified NK cell expresses an increased level of PDGFRβ having at least 90% sequence identity to SEQ ID NO:9 relative to a non-genetically-modified NK cell. In embodiments, the genetically-modified NK cell expresses an increased level of PDGFRβ having at least 95% sequence identity to SEQ ID NO:9 relative to a non-genetically-modified NK cell. In embodiments, the genetically-modified NK cell expresses an increased level of PDGFRβ having SEQ ID NO:9 relative to a non-genetically-modified NK cell. In embodiments, the genetically-modified NK cell comprises an exogenous nucleic acid that expresses an increased level of PDGFRβ having at least 85% sequence identity to SEQ ID NO:9 relative to a non-genetically-modified NK cell. In embodiments, the genetically-modified NK cell comprises an exogenous nucleic acid that expresses an increased level of PDGFRβ having at least 90% sequence identity to SEQ ID NO:9 relative to a non-genetically-modified NK cell. In embodiments, the genetically-modified NK cell comprises an exogenous nucleic acid that expresses an increased level of PDGFRβ having at least 95% sequence identity to SEQ ID NO:9 relative to a non-genetically-modified NK cell. In embodiments, the genetically-modified NK cell comprises an exogenous nucleic acid that expresses an increased level of PDGFRβ having SEQ ID NO:9 relative to a non-genetically-modified NK cell. In embodiments, the genetically-modified NK cell constitutively expresses PDGFRβ having at least 85% sequence identity to SEQ ID NO:9. In embodiments, the genetically-modified NK cell constitutively expresses PDGFRβ having at least 90% sequence identity to SEQ ID NO:9. In embodiments, the genetically-modified NK cell constitutively expresses PDGFRβ having at least 95% sequence identity to SEQ ID NO:9. In embodiments, the genetically-modified NK cell constitutively expresses PDGFRβ having SEQ ID NO:9.

Provided herein is a genetically-modified NK cell that expresses PDGFRβ having at least 85% sequence identity to SEQ ID NO:10. In embodiments, the genetically-modified NK cell expresses PDGFRβ having at least 90% sequence identity to SEQ ID NO:10. In embodiments, the genetically-modified NK cell expresses PDGFRβ having at least 95% sequence identity to SEQ ID NO:10. In embodiments, the genetically-modified NK cell expresses PDGFRβ having SEQ ID NO:10. In embodiments, the genetically-modified NK cell comprises an exogenous nucleic acid that expresses PDGFRβ having at least 85% sequence identity to SEQ ID NO:10. In embodiments, the genetically-modified NK cell comprises an exogenous nucleic acid that expresses PDGFRβ having at least 90% sequence identity to SEQ ID NO:10. In embodiments, the genetically-modified NK cell comprises an exogenous nucleic acid that expresses PDGFRβ having at least 95% sequence identity to SEQ ID NO:10. In embodiments, the genetically-modified NK cell comprises an exogenous nucleic acid that expresses PDGFRβ having SEQ ID NO:10. In embodiments, the genetically-modified NK cell expresses an increased level of PDGFRβ having at least 85% sequence identity to SEQ ID NO:10 relative to a non-genetically-modified NK cell. In embodiments, the genetically-modified NK cell expresses an increased level of PDGFRβ having at least 90% sequence identity to SEQ ID NO:10 relative to a non-genetically-modified NK cell. In embodiments, the genetically-modified NK cell expresses an increased level of PDGFRβ having at least 95% sequence identity to SEQ ID NO:10 relative to a non-genetically-modified NK cell. In embodiments, the genetically-modified NK cell expresses an increased level of PDGFRβ having SEQ ID NO:10 relative to a non-genetically-modified NK cell. In embodiments, the genetically-modified NK cell comprises an exogenous nucleic acid that expresses an increased level of PDGFRβ having at least 85% sequence identity to SEQ ID NO:10 relative to a non-genetically-modified NK cell. In embodiments, the genetically-modified NK cell comprises an exogenous nucleic acid that expresses an increased level of PDGFRβ having at least 90% sequence identity to SEQ ID NO:10 relative to a non-genetically-modified NK cell. In embodiments, the genetically-modified NK cell comprises an exogenous nucleic acid that expresses an increased level of PDGFRβ having at least 95% sequence identity to SEQ ID NO:10 relative to a non-genetically-modified NK cell. In embodiments, the genetically-modified NK cell comprises an exogenous nucleic acid that expresses an increased level of PDGFRβ having SEQ ID NO:10 relative to a non-genetically-modified NK cell. In embodiments, the genetically-modified NK cell constitutively expresses PDGFRβ having at least 85% sequence identity to SEQ ID NO:10. In embodiments, the genetically-modified NK cell constitutively expresses PDGFRβ having at least 90% sequence identity to SEQ ID NO:10. In embodiments, the genetically-modified NK cell constitutively expresses PDGFRβ having at least 95% sequence identity to SEQ ID NO:10. In embodiments, the genetically-modified NK cell constitutively expresses PDGFRβ having SEQ ID NO:10.

Provided herein is a genetically-modified NK cell that express expresses platelet-derived growth factor (PDGF) and platelet-derived growth factor receptor (PDGFR). In embodiments, the genetically-modified NK cell comprises an exogenous nucleic acid that expresses PDGF and an exogenous nucleic acid that expresses PDGFR. In embodiments, the genetically-modified NK cell expresses an increased level of PDGF relative to a non-genetically-modified NK cell and an increased level of PDGFR relative to a non-genetically-modified NK cell. In embodiments, the genetically-modified NK cell comprises an exogenous nucleic acid that expresses an increased level of PDGF relative to a non-genetically-modified NK cell and an exogenous nucleic acid that expresses an increased level of PDGFR relative to a non-genetically-modified NK cell. In embodiments, the genetically-modified NK cell constitutively expresses PDGF and PDGFR.

Provided herein is a genetically-modified NK cell that expresses platelet-derived growth factor D (PDGF-D) and platelet-derived growth factor receptor beta (PDGFRβ). In embodiments, the genetically-modified NK cell comprises an exogenous nucleic acid that expresses PDGF-D and an exogenous nucleic acid that expresses PDGFRβ. In embodiments, the genetically-modified NK cell expresses an increased level of PDGF-D relative to a non-genetically-modified NK cell and an increased level of PDGFRβ relative to a non-genetically-modified NK cell. In embodiments, the genetically-modified NK cell comprises an exogenous nucleic acid that expresses an increased level of PDGF-D relative to a non-genetically-modified NK cell and an exogenous nucleic acid that expresses an increased level of PDGFRβ relative to a non-genetically-modified NK cell. In embodiments, the genetically-modified NK cell constitutively expresses PDGF-D and PDGFRβ. In embodiments, the PDGF-D has at least 85% sequence identity to SEQ ID NO:7 and the PDGFRβ has at least 85% sequence identity to SEQ ID NO:8, 9, or 10. In embodiments, the PDGF-D has at least 90% sequence identity to SEQ ID NO:7 and the PDGFRβ has at least 90% sequence identity to SEQ ID NO:8, 9, or 10. In embodiments, the PDGF-D has at least 95% sequence identity to SEQ ID NO:7 and the PDGFRβ has at least 95% sequence identity to SEQ ID NO:8, 9, or 10. In embodiments, the PDGF-D has SEQ ID NO:7 and the PDGFRβ has SEQ ID NO:8, 9, or 10. In embodiments, the PDGF-D has at least 85% sequence identity to SEQ ID NO:7 and the PDGFRβ has at least 85% sequence identity to SEQ ID NO:8. In embodiments, the PDGF-D has at least 90% sequence identity to SEQ ID NO:7 and the PDGFRβ has at least 90% sequence identity to SEQ ID NO:8. In embodiments, the PDGF-D has at least 95% sequence identity to SEQ ID NO:7 and the PDGFRβ has at least 95% sequence identity to SEQ ID NO:8. In embodiments, the PDGF-D has SEQ ID NO:7 and the PDGFRβ has SEQ ID NO:8. In embodiments, the PDGF-D has at least 85% sequence identity to SEQ ID NO:7 and the PDGFRβ has at least 85% sequence identity to SEQ ID NO:9. In embodiments, the PDGF-D has at least 90% sequence identity to SEQ ID NO:7 and the PDGFRβ has at least 90% sequence identity to SEQ ID NO:9. In embodiments, the PDGF-D has at least 95% sequence identity to SEQ ID NO:7 and the PDGFRβ has at least 95% sequence identity to SEQ ID NO:9. In embodiments, the PDGF-D has SEQ ID NO:7 and the PDGFRβ has SEQ ID NO:9. In embodiments, the PDGF-D has at least 85% sequence identity to SEQ ID NO:7 and the PDGFRβ has at least 85% sequence identity to SEQ ID NO:10. In embodiments, the PDGF-D has at least 90% sequence identity to SEQ ID NO:7 and the PDGFRβ has at least 90% sequence identity to SEQ ID NO:10. In embodiments, the PDGF-D has at least 95% sequence identity to SEQ ID NO:7 and the PDGFRβ has at least 95% sequence identity to SEQ ID NO:10. In embodiments, the PDGF-D has SEQ ID NO:7 and the PDGFRβ has SEQ ID NO:10.

The term “an increased level” of PDGF and/or PDGF-D (or embodiments thereof) relative to a non-genetically-modified NK cell means a level of PDGF and/or PDGFR that is at least 5% higher than the level of PDGF and/or PDGFR expressed by a non-genetically-modified NK cell. In embodiments, the increased level of PDGF and/or PDGFR is at least 10% higher than the level of PDGF and/or PDGFR expressed by a non-genetically-modified NK cell. In embodiments, the increased level of PDGF and/or PDGFR is at least 15% higher than the level of PDGF and/or PDGFR expressed by a non-genetically-modified NK cell. In embodiments, the increased level of PDGF and/or PDGFR is at least 20% higher than the level of PDGF and/or PDGFR expressed by a non-genetically-modified NK cell. In embodiments, the increased level of PDGF and/or PDGFR is at least 25% higher than the level of PDGF and/or PDGFR expressed by a non-genetically-modified NK cell. In embodiments, the increased level of PDGF and/or PDGFR is at least 30% higher than the level of PDGF and/or PDGFR expressed by a non-genetically-modified NK cell. In embodiments, the increased level of PDGF and/or PDGFR is at least 35% higher than the level of PDGF and/or PDGFR expressed by a non-genetically-modified NK cell. In embodiments, the increased level of PDGF and/or PDGFR is at least 40% higher than the level of PDGF and/or PDGFR expressed by a non-genetically-modified NK cell. In embodiments, the increased level of PDGF and/or PDGFR is at least 45% higher than the level of PDGF and/or PDGFR expressed by a non-genetically-modified NK cell. In embodiments, the increased level of PDGF and/or PDGFR is at least 50% higher than the level of PDGF and/or PDGFR expressed by a non-genetically-modified NK cell. In embodiments, the increased level of PDGF and/or PDGFR is at least 55% higher than the level of PDGF and/or PDGFR expressed by a non-genetically-modified NK cell. In embodiments, the increased level of PDGF and/or PDGFR is at least 60% higher than the level of PDGF and/or PDGFR expressed by a non-genetically-modified NK cell. In embodiments, the increased level of PDGF and/or PDGFR is at least 65% higher than the level of PDGF and/or PDGFR expressed by a non-genetically-modified NK cell. In embodiments, the increased level of PDGF and/or PDGFR is at least 70% higher than the level of PDGF and/or PDGFR expressed by a non-genetically-modified NK cell. In embodiments, the increased level of PDGF and/or PDGFR is at least 75% higher than the level of PDGF and/or PDGFR expressed by a non-genetically-modified NK cell. In embodiments, the increased level of PDGF and/or PDGFR is at least 80% higher than the level of PDGF and/or PDGFR expressed by a non-genetically-modified NK cell. In embodiments, the increased level of PDGF and/or PDGFR is at least 85% higher than the level of PDGF and/or PDGFR expressed by a non-genetically-modified NK cell. In embodiments, the increased level of PDGF and/or PDGFR is at least 90% higher than the level of PDGF and/or PDGFR expressed by a non-genetically-modified NK cell. In embodiments, the increased level of PDGF and/or PDGFR is at least 95% higher than the level of PDGF and/or PDGFR expressed by a non-genetically-modified NK cell. In embodiments, a non-genetically-modified NK cell is a NK cell that has not been genetically modified. In embodiments, a non-genetically-modified NK cell is a NK cell that has not been genetically modified to express PDGF and/or PDGFR. In embodiments, a non-genetically-modified NK cell for purposes of this embodiment is a NK cell that has been genetically-modified, but has not been genetically modified to express PDGF and/or PDGFR. In embodiments, a non-genetically-modified NK cell for purposes of this embodiment is a NK cell that has been genetically-modified to express one or more proteins selected from the group consisting of PD-L1, IL-15 (e.g., soluble IL-15), tEGFR protein, and a chimeric antigen receptor, but has not been genetically modified to express PDGF and/or PDGFR.

In embodiments, the NK cell is a CD56^{dim} NK cell. The term CD56^{dim} NK cell is used in accordance to its ordinary meaning in the art and refers to NK cells that express a lower density of CD56 antigen as compared to CD56^{bright} NK cells. CD56^{dim} NK cells are involved in natural and/or antibody-mediated cell cytotoxicity, and typically contain higher levels of perforin and granzyme A compared to CD56^{bright} NK cells

In embodiments, the NK cell is a human NK cell.

In embodiments, the genetically-modified NK cells expresses PDGF (or PDGF-D or embodiments thereof as described herein) and one or more proteins selected from the group consisting of PD-L1, IL-15, and truncated epidermal growth factor receptor (tEGFR) protein. In embodiments, the genetically-modified NK cells expresses PDGF (or PDGF-D or embodiments thereof as described herein), PD-L1, IL-15, and tEGFR protein. In embodiments, the genetically-modified NK cells expresses PDGF (or PDGF-D or embodiments thereof as described herein) and one or more proteins selected from the group consisting of PD-L1, soluble IL-15, and tEGFR protein. In embodiments, the genetically-modified NK cells expresses PDGF (or PDGF-D or embodiments thereof as described herein), PD-L1, soluble IL-15, and tEGFR protein. In embodiments, the genetically-modified NK cells expresses PDGF (or PDGF-D or embodiments thereof as described herein) and one or more proteins selected from the group consisting of PD-L1, IL-15, tEGFR protein, and a chimeric antigen receptor. In embodiments, the genetically-modified NK cells expresses PDGF (or PDGF-D or embodiments thereof as described herein), PD-L1, IL-15, tEGFR protein, and a chimeric antigen receptor. In embodiments, the genetically-modified NK cells expresses PDGF (or PDGF-D or embodiments thereof as described herein) and one or more proteins selected from the group consisting of PD-L1, soluble IL-15, tEGFR protein, and a chimeric antigen receptor. In embodiments, the genetically-modified NK cells expresses PDGF (or PDGF-D or embodiments thereof as described herein), PD-L1, soluble IL-15, tEGFR protein, and a chimeric antigen receptor.

In embodiments, the genetically-modified NK cells expresses PDGFR (or PDGFRβ or embodiments thereof as described herein) and one or more proteins selected from the group consisting of PD-L1, IL-15, and truncated epidermal growth factor receptor (tEGFR) protein. In embodiments, the genetically-modified NK cells expresses PDGFR (or PDGFRβ or embodiments thereof as described herein), PD-L1, IL-15, and tEGFR protein. In embodiments, the genetically-modified NK cells expresses PDGFR (or PDGFRβ or embodiments thereof as described herein) and one or more proteins selected from the group consisting of PD-L1, soluble IL-15, and tEGFR protein. In embodiments, the genetically-modified NK cells expresses PDGFR (or PDGFRβ or embodiments thereof as described herein), PD-L1, soluble IL-15, and tEGFR protein. In embodiments, the genetically-modified NK cells expresses PDGFR (or PDGFRβ or embodiments thereof as described herein) and one or more proteins selected from the group consisting of PD-L1, IL-15, tEGFR protein, and a chimeric antigen receptor. In embodiments, the genetically-modified NK cells expresses PDGFR (or PDGFRβ or embodiments thereof as described herein), PD-L1, IL-15, tEGFR protein, and a chimeric antigen receptor. In embodiments, the genetically-modified NK cells expresses PDGFR (or PDGFRβ or embodiments thereof as described herein) and one or more proteins selected from the group consisting of PD-L1, soluble IL-15, tEGFR protein, and a chimeric antigen receptor. In embodiments, the genetically-modified NK cells expresses PDGFR (or PDGFRβ or embodiments thereof as described herein), PD-L1, soluble IL-15, tEGFR protein, and a chimeric antigen receptor.

In embodiments, the genetically-modified NK cells expresses PDGF and PDGFR (including embodiments of each as described herein) and one or more proteins selected from the group consisting of PD-L1, IL-15, and truncated epidermal growth factor receptor (tEGFR) protein. In embodiments, the genetically-modified NK cells expresses PDGF and/or PDGFR (including embodiments of each as described herein), PD-L1, IL-15, and tEGFR protein. In embodiments, the genetically-modified NK cells expresses PDGF and PDGFR (including embodiments of each as described herein) and one or more proteins selected from the group consisting of PD-L1, soluble IL-15, and tEGFR protein. In embodiments, the genetically-modified NK cells expresses PDGF and PDGFR (including embodiments of each as described herein), PD-L1, soluble IL-15, and tEGFR protein. In embodiments, the genetically-modified NK cells expresses PDGF and PDGFR (including embodiments of each as described herein) and one or more proteins selected from the group consisting of PD-L1, IL-15, tEGFR protein, and a chimeric antigen receptor. In embodiments, the genetically-modified NK cells expresses PDGF and PDGFR (including embodiments of each as described herein), PD-L1, IL-15, tEGFR protein, and a chimeric antigen receptor. In embodiments, the genetically-modified NK cells expresses PDGF and PDGFR (including embodiments of each as described herein) and one or more proteins selected from the group consisting of PD-L1, soluble IL-15, tEGFR protein, and a chimeric antigen receptor. In embodiments, the genetically-modified NK cells expresses PDGF and PDGFR (including embodiments of each as described herein), PD-L1, soluble IL-15, tEGFR protein, and a chimeric antigen receptor.

In embodiments, the NK cell is a PD-L1(+) NK cell. In embodiments, the NK cell expresses PD-L1. In embodiments, the NK cell comprises an exogenous nucleic acid that expresses PD-L1. In embodiments, the genetically-modified NK cell constitutively expresses PD-L1. In embodiments, the NK cell expresses cell surface PD-L1. In embodiments, the NK cell comprises an exogenous nucleic acid that expresses cell surface PD-L1.

In embodiments, the NK cell expresses IL-15. In embodiments, the NK cell comprises an exogenous nucleic acid that expresses IL-15. In embodiments, the NK cell constitutively expresses IL-15. In embodiments, the NK cell is an activated cord blood NK cell that has been genetically modified to constitutively express IL-15. In embodiments, the IL-15 is recombinant IL-15. In embodiments, the IL-15 is exogenous to the NK cell.

In embodiments, the NK cell expresses soluble IL-15. In embodiments, the NK cell comprises an exogenous nucleic acid that expresses soluble IL-15. In embodiments, the NK cell constitutively expresses soluble IL-15. In embodiments, the NK cell is an activated cord blood NK cell that has been genetically modified to constitutively express soluble IL-15. In embodiments, the IL-15 is recombinant IL-15. In embodiments, the IL-15 is exogenous to the NK cell.

In embodiments, the control is an NK cell that has not been contacted with IL-15. In embodiments, the control is an NK cell that has not been contacted with exogenous IL-15. In embodiments, the control is an NK cell that does not express an exogenous IL-15. In embodiments, the control is an NK cell that does not express a recombinant IL-15.

In embodiments, the NK cell expresses truncated epidermal growth factor receptor (tEGFR) protein. In embodiments, the NK cell comprises an exogenous nucleic acid that expresses truncated epidermal growth factor receptor (tEGFR) protein. In embodiments, the NK cell constitutively expresses tEGFR protein.

In embodiments, the NK cell is derived from umbilical cord blood NK cells. In embodiments, the umbilical cord blood NK cells were incubated with IL-12 and IL-18. In embodiments, the umbilical cord blood NK cells were incubated with IL-2, IL-12, IL-15, IL-18, or a combination of two or more thereof. In embodiments, the umbilical cord blood NK cells were incubated with IL-2, IL-12, IL-15 and IL-18.

In embodiments, the genetically-modified NK cell expresses a chimeric antigen receptor. In embodiments, the genetically-modified NK cell comprises an exogenous nucleic acid that expresses a chimeric antigen receptor. In embodiments, the target of the chimeric antigen receptor is CD7, CD19, CD22, CD33, BCMA, MUC1, PSMA, mesothelin, NKG2D, ROBO1, or HER2. In embodiments, the target of the chimeric antigen receptor is CD7, CD22, CD33, BCMA, MUC1, PSMA, mesothelin, NKG2D, ROBOT, or HER2. Chimeric antigen receptors are described, for example, by Yilmaz et al, J. Hematol Oncol, 13:168 (2020); Monsour et al, Success and Challenges of NK Immunotherapy, Chapter 12, pages 213-220 (2021); and WO 2022/115421, the disclosures of which are incorporated by reference herein in their entirety.

In embodiments, the NK cell includes a chimeric antigen receptor (CAR). In embodiments, the NK cell includes a CD7, CD19, CD22, CD33, BCMA, MUC1, PSMA, mesothelin, NKG2D, ROBO1, or HER2 chimeric antigen receptor (CAR). In embodiments, the NK cell includes a CD7, CD22, CD33, BCMA, MUC1, PSMA, mesothelin, NKG2D, ROBOT, or HER2 chimeric antigen receptor (CAR). In embodiments, the NK cell does not include a CD19 chimeric antigen receptor (CAR). Chimeric antigen receptors are described, for example, by Yilmaz et al, J. Hematol Oncol, 13:168 (2020); Monsour et al, Success and Challenges of NK Immunotherapy, Chapter 12, pages 213-220 (2021); and WO 2022/115421, the disclosures of which are incorporated by reference herein in their entirety.

Provided herein are a population of NK cells comprising a plurality of the genetically-modified NK cells (e.g., NK cells) described herein, including embodiments thereof

### Pharmaceutical Compositions

The compositions provided herein include pharmaceutical compositions including the NK cells provided herein including embodiments thereof. Thus, in an aspect is provided a pharmaceutical composition including a therapeutically effective amount of an NK cell as provided herein including embodiments thereof and a pharmaceutically acceptable excipient.

Provided herein are pharmaceutical compositions comprising a genetically-modified NK cell as described herein, including embodiments thereof, and a pharmaceutically acceptable excipient. Provided herein are pharmaceutical compositions comprising a population of genetically-modified NK cells as described herein, including embodiments thereof, and a pharmaceutically acceptable excipient.

### Methods of Making

Provided herein are methods of producing PDGF and/or PDGFR expressing NK cells. NK cells produced by the methods provided herein are contemplated to have enhanced survival capabilities. Thus, in an aspect is provided a method of generating a natural killer (NK) cell capable of expressing platelet-derived growth factor (PDGF), the method including contacting the NK cell with IL-15. In another aspect is provided a method of generating a natural killer (NK) cell capable of expressing platelet-derived growth factor receptor (PDGFR), the method including contacting the NK cell with IL-15. In embodiments, the IL-15 is recombinant IL-15. In embodiments, the IL-15 is exogenous to the NK cell.

### Methods of Expanding NK Cells

Provided herein are methods of expanding a population of NK cells comprising contacting NK cells with an effective amount of platelet-derived growth factor (PDGF). In embodiments, the methods comprise contacting NK cells with an effective amount of PDGF and an effective amount of one or more cytokines selected from the group consisting of IL-2, IL-12, IL-15, and IL-18. In embodiments, the methods comprise contacting NK cells with an effective amount of PDGF, IL-2, IL-12, IL-15, and IL-18. In embodiments, the method further comprises incubating the NK cells with K562 feeder cells. In embodiments, the K562 feeder cells express membrane-bound IL-21 and CD-137L and exogenous IL-2. In embodiments, the NK cells are not genetically modified. In embodiments, the NK cells are not genetically modified to express PDGF. In embodiments, the NK cells are not genetically modified to express PDGFR. In embodiments, the NK cells are not genetically modified to express PDGF or PDGFR. In embodiments, the NK cells are genetically modified to express one or more proteins selected from the group consisting of PD-L1, IL-15, tEGFR protein, and a chimeric antigen receptor. In embodiments, the NK cells are genetically modified to express one or more proteins selected from the group consisting of PD-L1, IL-15, tEGFR protein, and a chimeric antigen receptor, but are not genetically-modified to express PDGF and/or PDGFR. Methods of expanding NK cells are known in the art and described, for example, by Cho et al, Korean J. Lab Med, 29(2):89-96 (2009) and Monsour et al, Success and Challenges of NK Immunotherapy, Chapter 12, pages 213-220 (2021), the disclosures of which are incorporated by reference herein in their entirety.

Provided herein are methods of expanding a population of NK cells comprising contacting NK cells with an effective amount of platelet-derived growth factor D (PDGF-D). In embodiments, the methods comprise contacting NK cells with an effective amount of PDGF-D and an effective amount of one or more cytokines selected from the group consisting of IL-2, IL-12, IL-15, and IL-18. In embodiments, the methods comprise contacting NK cells with an effective amount of PDGF-D, IL-2, IL-12, IL-15, and IL-18. In embodiments, the method further comprises incubating the NK cells with K562 feeder cells. In embodiments, the NK cells are not genetically modified. In embodiments, the NK cells are not genetically modified to express PDGF-D. In embodiments, the NK cells are not genetically modified to express PDGFRβ. In embodiments, the NK cells are not genetically modified to express PDGF-D or PDGFRβ. In embodiments, the NK cells are genetically modified to express one or more proteins selected from the group consisting of PD-L1, IL-15, tEGFR protein, and a chimeric antigen receptor. In embodiments, the NK cells are genetically modified to express one or more proteins selected from the group consisting of PD-L1, IL-15, tEGFR protein, and a chimeric antigen receptor, but are not genetically-modified to express PDGF-D and/or PDGFRβ. Methods of expanding NK cells are known in the art and described, for example, by Cho et al, Korean J. Lab Med, 29(2):89-96 (2009) and Monsour et al, Success and Challenges of NK Immunotherapy, Chapter 12, pages 213-220 (2021), the disclosures of which are incorporated by reference herein in their entirety.

### Methods of Treatment

The NK cells provided herein including embodiments thereof, are contemplated as providing effective treatments for cancer. Thus, in an aspect is provided a method of treating cancer in a subject in need thereof, the method including administering to the subject an effective amount of a population of the NK cell provided herein including embodiments thereof. In embodiments, the methods of treating cancer comprise administering to a subject an effective amount of the genetically-modified NK cells described herein, including embodiments thereof. In embodiments, the cancer is lung cancer, neuroblastoma, glioma, myelodysplastic syndrome, leukemia, lymphoma, liver cancer, prostate cancer, pancreatic cancer, gastric cancer, head and neck cancer, multiple myeloma, biliary tract cancer, ovarian cancer, melanoma, or colorectal cancer. In embodiments, the cancer is lung cancer. In embodiments, the cancer is neuroblastoma. In embodiments, the cancer is glioma. In embodiments, the cancer is myelodysplastic syndrome. In embodiments, the cancer is liver cancer. In embodiments, the cancer is prostate cancer. In embodiments, the cancer is pancreatic cancer. In embodiments, the cancer is gastric cancer. In embodiments, the cancer is head and neck cancer. In embodiments, the cancer is multiple myeloma. In embodiments, the cancer is biliary tract cancer. In embodiments, the cancer is ovarian cancer. In embodiments, the cancer is melanoma. In embodiments, the cancer is colorectal cancer. In embodiments, the cancer is non-small cell lung cancer. In embodiments, the cancer is leukemia. In embodiments, the cancer is myeloid leukemia. In embodiments, the cancer is acute myeloid leukemia. In embodiments, the cancer is chronic myeloid leukemia. In embodiments, the cancer is lymphoblastic leukemia. In embodiments, the cancer is lymphoma. In embodiments, the cancer is B cell lymphoma. In embodiments, the cancer is non-Hodgkin lymphoma. In embodiments, the patient is refractory to chemotherapy. In embodiments, the patient is refractory to a PD-1 inhibitor and/or a PD-L1 inhibitor.

In embodiments, the methods of treating cancer further comprise administering to the patient an effective amount of a checkpoint inhibitor. In embodiments, the checkpoint inhibitor is a PD-1 inhibitor, a PD-L1 inhibitor, or a CTLA-4 inhibitor. In embodiments, the checkpoint inhibitor is a PD-1 inhibitor. In embodiments, the PD-1 inhibitor is pembrolizumab, nivolumab, cemiplimab, dostarlimab, JTX-4014, spartalizumab, camrelizumab, sintilimab, tislelizumab, toripalimab, retifanlimab, AMP-224, or AMP-514. In embodiments, the checkpoint inhibitor is a PD-L1 inhibitor. In embodiments, the PD-L1 inhibitor is atezolizumab, avelumab, durvalumab, envafolimab, cosibelimab, AUNP12, CA-170, or BMS-986189. In embodiments, the checkpoint inhibitor is a CTLA-4 inhibitor. In embodiments, the CTLA-4 inhibitor is ipilimumab.

In embodiments, the NK cell is administered intraperitoneally, intratumorally, intravenously, intrathecally, or intrapleurally. In embodiments, the NK cell is administered intraperitoneally. In embodiments, the NK cell is administered intratumorally. In embodiments, the NK cell is administered intravenously. In embodiments, the NK cell is administered intrathecally. In embodiments, the NK cell is administered intrapleurally.

### Embodiments 1-52

Embodiment 1. A genetically-modified NK cell that expresses: (i) platelet-derived growth factor (PDGF); (ii) platelet-derived growth factor receptor (PDGFR); or (iii) PDGF and PDGFR.

Embodiment 2. The genetically-modified NK cell of Embodiment 1, wherein the PDGF is platelet-derived growth factor D (PDGF-D) and wherein the PDGFR is platelet-derived growth factor receptor beta (PDGFRβ).

Embodiment 3. The genetically-modified NK cell of Embodiment 1 or 2 that expresses PDGF.

Embodiment 4. The genetically-modified NK cell of Embodiment 1 or 2 that expresses PDGFR.

Embodiment 5. The genetically-modified NK cell of Embodiment 1 or 2 that expresses PDGF and PDGFR.

Embodiment 6. The genetically-modified NK cell of any one of Embodiments 1-3 and 5, wherein the genetically-modified NK cell expresses an increased level of PDGF relative to a non-genetically-modified NK cell.

Embodiment 7. The genetically-modified NK cell of any one of Embodiments 1, 2, and 4-6, wherein the genetically-modified NK cell expresses an increased level of PDGFR relative to a non-genetically-modified NK cell.

Embodiment 8. The genetically-modified NK cell of any one of Embodiments 1-3, 5, and 6, wherein the genetically-modified NK cell comprises an exogenous nucleic acid that expresses PDGF.

Embodiment 9. The genetically-modified NK cell of any one of Embodiments 1, 2, and 4-8, wherein the genetically-modified NK cell comprises an exogenous nucleic acid that expresses PDGFR.

Embodiment 10. The genetically-modified NK cell of Embodiment 8 or 9, wherein the exogenous nucleic acid is under inducible control.

Embodiment 11. The genetically-modified NK cell of any one of Embodiments 1 to 10, wherein the genetically-modified NK cell constitutively expresses PDGF and/or PDGFR.

Embodiment 12. The genetically-modified NK cell of any one of Embodiments 1 to 11, wherein the genetically-modified NK cell is a CD56^{dim} NK cell.

Embodiment 13. The genetically-modified NK cell of any one of Embodiments 1 to 12, wherein the genetically-modified NK cell is a human NK cell.

Embodiment 14. The genetically-modified NK cell of any one of Embodiments 1 to 13, wherein the genetically-modified NK cell expresses PD-L1.

Embodiment 15, The genetically-modified NK cell of Embodiment 14, wherein the genetically-modified NK cell comprises an exogenous nucleic acid that expresses PD-L1.

Embodiment 16. The genetically-modified NK cell of Embodiment 14 or 15, wherein the genetically-modified NK cell constitutively expresses PD-L1.

Embodiment 17. The genetically-modified NK cell of any one of Embodiments 1 to 16, wherein the genetically-modified NK cell expresses IL-15.

Embodiment 18. The genetically-modified NK cell of Embodiment 17, wherein the genetically-modified NK cell comprises an exogenous nucleic acid that expresses IL-15.

Embodiment 19. The genetically-modified NK cell of Embodiment 17 or 18, wherein the genetically-modified NK cell constitutively expresses IL-15.

Embodiment 20. The genetically-modified NK cell of any one of Embodiments 17to 19, wherein the IL-15 is soluble IL-15.

Embodiment 21. The NK cell of any one of Embodiments 1 to 20, wherein the genetically-modified NK cell expresses truncated epidermal growth factor receptor (tEGFR) protein.

Embodiment 22. The genetically-modified NK cell of Embodiment 22, wherein the genetically-modified NK cell comprises an exogenous nucleic acid that expresses tEGFR protein.

Embodiment 23. The genetically-modified NK cell of Embodiment 21 or 22, wherein the genetically-modified NK cell constitutively expresses tEGFR protein.

Embodiment 24. The genetically-modified NK cell of any one of Embodiments 1 to 23, wherein the genetically-modified NK cell comprises an exogenous nucleic acid that expresses a chimeric antigen receptor.

Embodiment 25. The genetically-modified NK cell of any one of Embodiments 1 to 24, wherein the genetically-modified NK cell is an activated cord blood NK cell.

Embodiment 26. The NK cell of any one of Embodiments 1 to 25, wherein the genetically-modified NK cell is derived from umbilical cord blood NK cells.

Embodiment 27. The NK cell of Embodiment 26, wherein the umbilical cord blood NK cells were incubated with IL-12, IL-15, IL-18, or a combination of two or more thereof.

Embodiment 28. The NK cell of any one of Embodiments 1 to 27, wherein the genetically-modified NK cell comprises a chimeric antigen receptor (CAR).

Embodiment 29. The NK cell of any one of Embodiments 1 to 28, wherein the genetically-modified NK cell does not comprise a CD19 chimeric antigen receptor (CAR).

Embodiment 30. A pharmaceutical composition comprising the genetically-modified NK cell of any one of Embodiments 1 to 29.

Embodiment 31. A population of genetically-modified NK cells comprising a plurality of the genetically-modified NK cell of any one of Embodiments 1 to 29.

Embodiment 32. A pharmaceutical composition comprising the population of natural killer cells of Embodiment 31.

Embodiment 33. A method of treating cancer in a subject in need thereof, the method comprising administering to the subject an effective amount of a population of the genetically-modified NK cell of any one of Embodiments 1 to 29; the pharmaceutical composition of Embodiment 30 or 32; or the population of genetically-modified NK cells of Embodiment 31.

Embodiment 34. The method of Embodiment 33, wherein the cancer is lung cancer, neuroblastoma, glioma, myelodysplastic syndrome, leukemia, lymphoma, liver cancer, prostate cancer, pancreatic cancer, gastric cancer, head and neck cancer, multiple myeloma, biliary tract cancer, ovarian cancer, melanoma, or colorectal cancer.

Embodiment 35. The method of Embodiment 34, wherein the lung cancer is non-small cell lung cancer.

Embodiment 36. The method of Embodiment 34, wherein the cancer is leukemia.

Embodiment 37. The method of Embodiment 36, wherein the leukemia is acute myeloid leukemia, chronic myeloid leukemia, or lymphoblastic leukemia.

Embodiment 38, The method of Embodiment 34, wherein the cancer is lymphoma.

Embodiment 39. The method of Embodiment 38, wherein the lymphoma is B cell lymphoma or non-Hodgkin lymphoma.

Embodiment 40. The method of any one of Embodiments 33 to 39, wherein the patient is refractory to chemotherapy.

Embodiment 41. The method of any one of Embodiments 33 to 40, wherein the patient is refractory to a PD-1 inhibitor and/or a PD-L1 inhibitor.

Embodiment 42. The method of any one of Embodiments 33 to 39, further comprising administering to the patient an effective amount of a checkpoint inhibitor.

Embodiment 43. The method of Embodiment 42, wherein the checkpoint inhibitor is a PD-1 inhibitor.

Embodiment 44. The method of Embodiment 43, wherein the PD-1 inhibitor is pembrolizumab, nivolumab, cemiplimab, dostarlimab, JTX-4014, spartalizumab, camrelizumab, sintilimab, tislelizumab, toripalimab, retifanlimab, AMP-224, or AMP-514.

Embodiment 45. The method of Embodiment 42, wherein the checkpoint inhibitor is a PD-L1 inhibitor.

Embodiment 46. The method of Embodiment 45, wherein the PD-L1 inhibitor is atezolizumab, avelumab, durvalumab, envafolimab, cosibelimab, AUNP12, CA-170, or BMS-986189.

Embodiment 47. The method of Embodiment 42, wherein the checkpoint inhibitor is a CTLA-4 inhibitor.

Embodiment 48. The method of Embodiment 47, wherein the CTLA-4 inhibitor is ipilimumab.

Embodiment 49, A method of expanding a population of natural killer cells in vitro, the method comprising contacting the NK cells with an effective amount of platelet-derived growth factor D (PDGF-D).

Embodiment 50. The method of Embodiment 49, further comprising contacting the NK cells with an effective amount of IL-2, IL-12, IL-15, IL-18, or a combination of two or more thereof

Embodiment 51. The method of Embodiment 49 or 50, further comprising incubating the NK cells with K562 feeder cells.

Embodiment 52. The method of Embodiment 51, wherein the K562 feeder cells express membrane-bound IL-21 and CD-137L and exogenous IL-2.

### Embodiments P1-P24

Embodiment P1. A natural killer (NK) cell capable of expressing platelet-derived growth factor (PDGF).

Embodiment P2. A natural kill (NK) cell capable of expressing an increased level of platelet-derived growth factor (PDGF) relative to a control.

Embodiment P3. The NK cell of Embodiment P1 or P2, wherein the PDGF is PDGF-D

Embodiment P4. A natural killer (NK) cell capable of expressing platelet-derived growth factor receptor (PDGFR).

Embodiment P5. A natural killer (NK) cell capable of expressing an increased level of platelet-derived growth factor receptor (PDGFR) relative to a control.

Embodiment P6. The NK cell of Embodiment P4 or P5, wherein the PDGFR is PDGFR (3.

Embodiment P7. The NK cell of any one of Embodiments P1 to P6, wherein the NK cell is a CD56^{dim} NK cell.

Embodiment P8. The NK cell of any one of Embodiments P1 to P7, wherein said NK cell is a human NK cell.

Embodiment P9. The NK cell of any one of Embodiments P1 to P8, wherein the NK cell is a PD-L1(+) NK cell.

Embodiment P10. The NK cell of any one of Embodiments P1 to P9, wherein the NK cell expresses soluble IL-15.

Embodiment P11. The NK cell of Embodiment P10, wherein the NK cell constitutively expresses soluble IL-15.

Embodiment P12. The NK cell of any one of Embodiments P1 to P11, wherein the NK cell is an activated cord blood NK cell that has been genetically modified to constitutively express soluble IL-15.

Embodiment P13. The NK cell of any one of Embodiments P10 to P12, wherein the IL-15 is a recombinant IL-15.

Embodiment P14. The NK cell of any one of Embodiments P1 to P13, wherein the NK cell expresses cell surface PD-L1.

Embodiment P15. The NK cell of any one of Embodiments P1 to P14, wherein the NK cell expresses truncated epidermal growth factor receptor (tEGFR) protein.

Embodiment P16. The NK cell of any one of Embodiments P1 to P15, wherein the NK cell is derived from umbilical cord blood NK cells.

Embodiment P17. The NK cell of Embodiment P16, wherein the umbilical cord blood NK cells were incubated with IL-12 and IL-18.

Embodiment P18. The NK cell of any one of Embodiments P1 to P17, wherein the NK cell comprises a chimeric antigen receptor (CAR).

Embodiment P19. The NK cell of any one of Embodiments P1 to P17, wherein the NK cell does not comprise a CD19 chimeric antigen receptor (CAR).

Embodiment P20. A method of generating a natural killer (NK) cell capable of expressing platelet-derived growth factor (PDGF), the method comprising contacting the NK cell with IL-15.

Embodiment P21. A method of generating a natural killer (NK) cell capable of expressing platelet-derived growth factor receptor (PDGFR), the method comprising contacting the NK cell with IL-15.

Embodiment P22. The method of Embodiment P20 or P21, wherein the IL-15 is recombinant IL-15.

Embodiment P23. The method of Embodiment P20 or P21, wherein the IL-15 is exogenous to the NK cell.

Embodiment P24. A method of treating cancer in a subject in need thereof, the method comprising administering to the subject an effective amount of a population of the NK cell of any one of Embodiments P1 to P19.

### EXAMPLES

### Example 1

The axis of platelet-derived growth factor (PDGF) and PDGF receptor-beta (PDGFRβ) plays prominent roles in cell growth and motility. In addition, PDGF-D enhances human natural killer (NK) cell effector functions when binding to the NKp44 receptor. Resting NK cells express no PDGFRβ and only a low level of PDGF-D, but both are significantly upregulated by IL-15, via the NF-κB signaling pathway, to promote cell survival in an autocrine manner. Both ectopic and IL-15-induced expression of PDGFRβ improves NK cell survival in response to treatment with PDGF-D.

In this study, we report a previously unknown role of PDGF-D: regulation of IL-15-mediated cell survival, not effector functions in human NK cells, that is dependent on PDGFRβ but independent of NKp44. The results indicate that the PDGF-DPDGFRβ signaling pathway is a new mechanism by which IL-15 selectively regulates the survival of human NK cells without modulating their effector functions.

### Example 2

### PDGF-D-PDGFRβ Signaling Enhances IL-15-Mediated Human Natural Killer Cell Survival

The axis of platelet-derived growth factor (PDGF) and PDGF receptor-beta (PDGFRβ) plays prominent roles in cell growth and motility. In addition, PDGF-D enhances human natural killer (NK) cell effector functions when binding to the NKp44 receptor. Here, an additional but previously unknown role of PDGF-D is described, whereby it mediates interleukin-15 (IL-15)-induced human NK cell survival but not effector functions via its binding to PDGFRβ but independent of its binding to NKp44. Resting NK cells express no PDGFRβ and only a low level of PDGF-D, but both are significantly upregulated by IL-15, via the NF-κB signaling pathway, to promote cell survival in an autocrine manner. Both ectopic and IL-15-induced expression of PDGFRβ improves NK cell survival in response to treatment with PDGF-D. Theses results indicate that the PDGF-D-PDGFRβ signaling pathway is a new mechanism by which IL-15 selectively regulates the survival of human NK cells without modulating their effector functions.

NK cells belong to a critical innate arm of host immunity against viral infection and malignancies. However, limited expansion and persistence of NK cells in vivo remain major challenges for NK cell-based therapy. Here, the data shows that PDGF-D-PDGFRβ signaling, a potent stimulator of cell growth and motility, activates NK cells in an autocrine manner and contributes to IL-15-mediated NK-cell survival but not effector functions, the latter of which were previously shown to depend on the binding of PDGF-D to the NKp44 receptor. Therefore, selectively introducing PDGF signaling into NK cells will benefit NK cell expansion and persistence and/or enhance effector function in NK cell-based immunotherapies.

### Results

### NK Cells Express High Levels of PDGFRβ Following IL-15 Stimulation

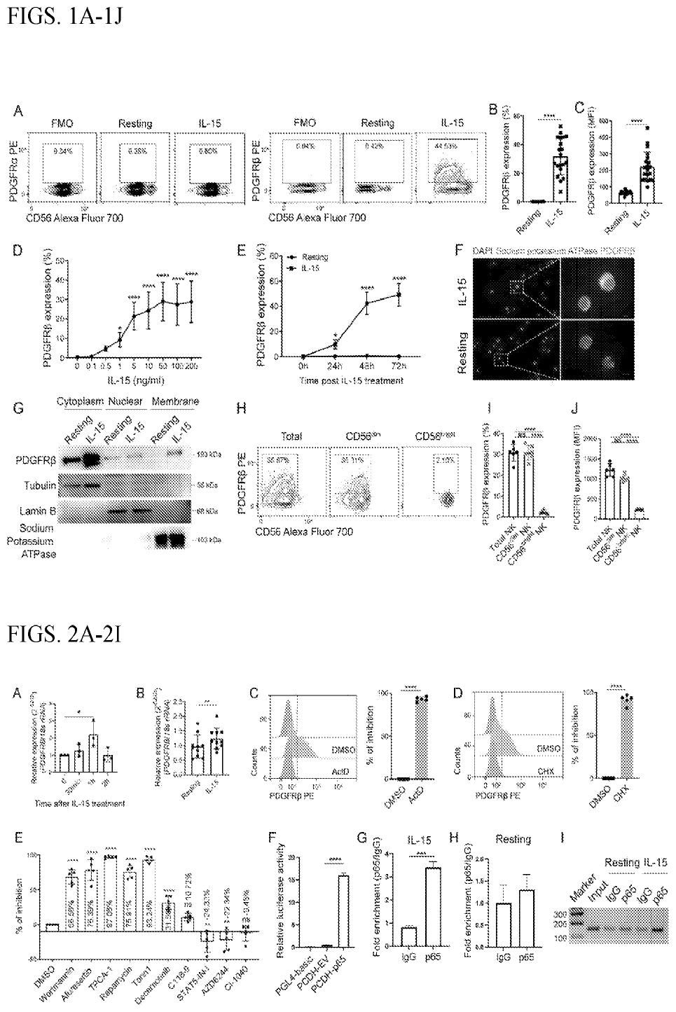

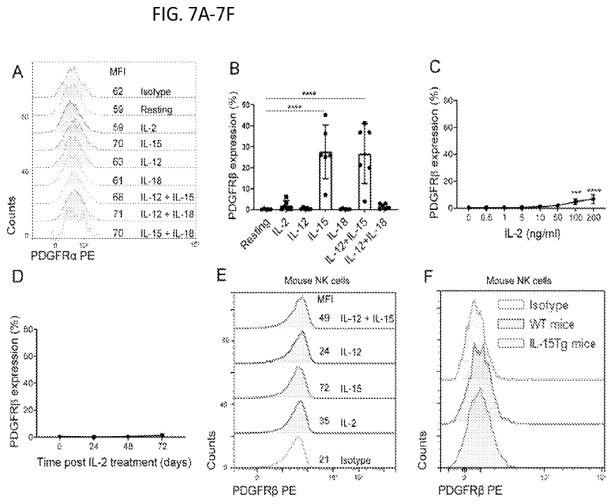

Previous studies suggested that NK cells and NK leukemia cells might express PDGF receptors (7, 8). However, the expression patterns have not been clearly described. Here, we enriched NK cells from peripheral blood mononuclear cells (PBMCs) of healthy donors (HCs) and examined PDGFRα and PDGFRβ expression by flow cytometry. Unexpectedly, PDGFRα and PDGFRβ could not be detected in resting primary NK cells from HCs (FIGS. 1A-1C and FIG. 7A). We then evaluated their levels after stimulation with different cytokines (IL-2, IL-12, IL-15, IL-18) or their combinations for 24 h. PDGFRα could not be induced by those cytokines or even their combinations (FIG. 7A). In contrast, IL-15 induced robust expression of PDGFRβ, and the effect was dose- and time-dependent (FIGS. 1D-1E). IL-12, IL-18, or the two combined was ineffective (FIG. 7B). IL-2, which shares the cognate receptors IL-2R13 and IL-2Ryc with IL-15, slightly but not significantly induced PDGFRβ expression (FIG. 7B) after 24 h culture at the concentration of 10 ng/ml. We further evaluated whether IL-2 could induce PDGFRβ expression at longer time points or higher doses. The results showed that the high doses of IL-2 (200 ng/ml) could only induce weak levels of PDGFRβ (FIG. 7C). A longer (up to 72 h) stimulation at the low dose (10 ng/ml) was ineffective (FIG. 7D). Our data on IL-2 are consistent with those from the prior study (6). Immunofluorescence confirmed that PDGFRβ was expressed on the membrane after IL-15 stimulation (FIG. 1F). Immunoblotting showed that PDGFRβ was enriched in the cytoplasm and translocated to the cell membrane following stimulation by IL-15 (FIG. 1G).

NK cells in peripheral blood are typically divided into CD56^{dim} and CD56^{bright} populations (9, 10). Interestingly, we found that IL-15 induced PDGFRβ expression only in CD56^{dim} NK cells and not CD56^{bright} NK cells (FIG. 1H-1J), indicating that PDGFRβ signaling affects only the former. To explore whether our findings in human NK cells could be reproduced in mouse NK cells, we purified splenic NK cells from C57BL/6 mice. However, the murine NK cells did not express PDGFRβ in the resting state or when stimulated with IL-2, IL-12, IL-15, or IL-12 plus IL-15 (FIG. 7E). We also could not detect any PDGFRβ after long-term IL-15 stimulation in our IL-15 transgenic mice (11, 12) (FIG. 7F). Thus. IL-15 induced PDGFRβ expression only in human NK cells. Taken together, our findings indicate that human NK cells express high levels of PDGFRβ after they are primed with IL-15.

### IL-15 Induces PDGFRβ Expression Through PI3K/AKT Signaling

To explore the mechanism by which IL-15 induces PDGFRβ expression in NK cells, we first measured mRNA levels of the PDGFRB gene. They increased significantly after IL-15 treatment (FIGS. 2A-2B), indicating that IL-15 induces PDGFRB expression at the transcriptional level. When we inhibited gene transcription with actinomycin D (ActD), IL-15 no longer induced PDGFRβ expression (FIG. 2C). In addition, blocking de novo protein synthesis with cycloheximide (CHX) completely inhibited the PDGFRβ expression induced by IL-15 (FIG. 2D).

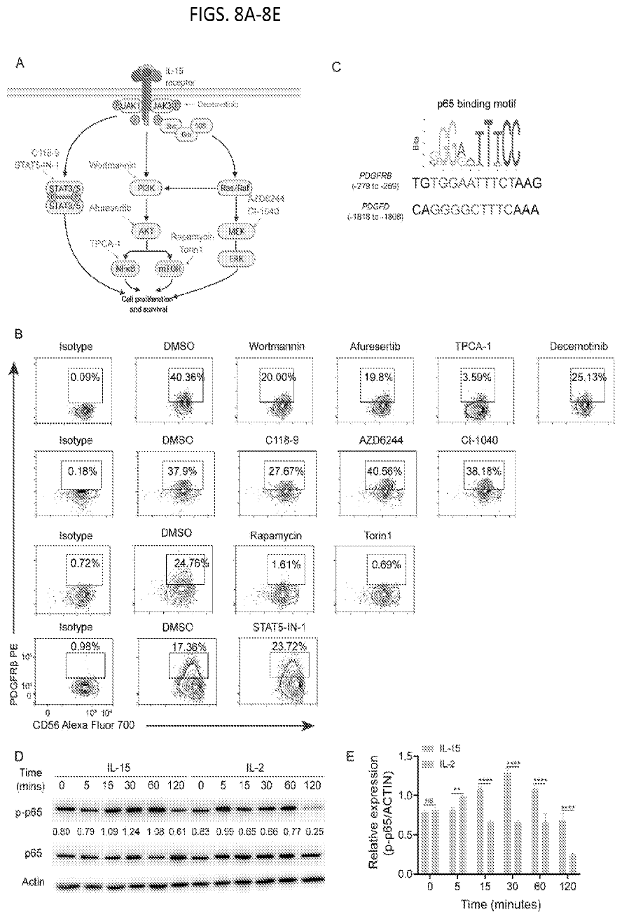

IL-15 signaling is mediated by at least three downstream signaling pathways in NK cells: MAPK/extracellular-signal-regulated kinase (ERK), PI3K/AKT, and signal transducer and activator of transcription 3/5 (STAT3/5) (FIG. 8A) (13). To determine which of these pathways regulates IL-15-mediated PDGFRβ induction, we pretreated NK cells with inhibitors that target specific pathway components and then stimulated the cells with IL-15(FIG. 8A). The PI3K inhibitor wortmannin and the pan-AKT kinase inhibitor afuresertib inhibited PDGFRβ expression by ˜70% (FIGS. 2E, 8B). Activated AKT activates downstream molecular proteins such as NF-κB and mTOR (13). We found that TPCA-1, an inhibitor of IkappaB kinase (an upstream component of NF-κB signaling) completely prevented IL-15 from stimulating PDGFRβ expression (FIGS. 2E, 8B). Treating NK cells with mTOR inhibitors, such as rapamycin or torin1, also dramatically and significantly blocked PDGFRβ expression by IL-15 (FIGS. 2E, 8B). In contrast, blocking Janus kinase 3 (JAK3) (decemotinib) signaling reduced PDGFRβ expression by approximately one third (FIGS. 2E, 8B); treatment with STAT signaling inhibitors (e.g. the STAT3 inhibitor C118-9, the STATS inhibitor STAT5-IN-1), or MAPK signaling inhibitors such as the MEK1 inhibitor AZD6244 or the MEK1/2 inhibitor CI-1040 produced either little inhibition or even promoted IL-15-induced PDGFRβ expression (FIGS. 2E, 8B). These data indicate that IL-15 likely induces PDGFRβ expression through the PI3K/AKT pathway. To strengthen this hypothesis, we showed that p65, a subunit of the NF-x13 complex that is downstream of the PI3K/AKT pathway, has a binding site in the promoter region of PDGFRB (FIG. 8C). Moreover, a luciferase reporter assay showed that p65 directly activated PDGFRB gene transcription (FIG. 2F). Chromatin immunoprecipitation (ChIP)-qPCR showed that IL-15-stimulated NK cells, but not resting NK cells, had significantly higher levels of p65associated with the PDGFRB promoter than the normal IgG control (FIGS. 2G-2I), indicating that p65 binds directly to the PDGFRB gene promoter in IL-15-stimulated NK cells. Of note, we found that IL-2 induced a level of phosphor (p)-p65 significantly lower than IL-15 at all the time points that we tested except for the 5 min early time point, when IL-2 induced a slightly higher level of p-p65 than IL-15 (FIGS. 8D-8E). This indicates that IL-2 is unable to strongly and durably induce the high levels of p-p65 that NK cells seem to require to upregulate PDGFRβ. Collectively, our data demonstrate that PDGFRβ expression in human NK cells is regulated by PI3K/AKT/NF-x13, which is downstream of IL-15 signaling.

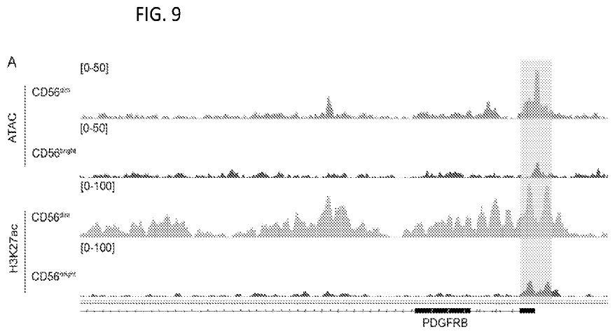

Transcriptional programs are regulated by chromatin accessibility, which can be indicated by transposase recognition and histone 3 lysine 27 acetylation (H3K27ac) (14, 15). High accessibility of chromatin to active gene promoters positively correlates with gene transcription (14, 16). To explore whether the accessibility of chromatin to the PDGFRB locus differs between CD56^{dim} and CD56^{bright} populations, we analyzed online datasets containing Assay for Transposase Accessible Chromatin with high-throughput sequencing (ATAC-seq) and H3K27ac ChIP-seq, both of which were performed in CD56^{bright} and CD56^{dim} NK cells (17). We found that both the ATAC-seq signal and enrichment of the H3K27ac modification in the promoter region of PDGFRB were higher in CD56^{dim} NK cells than in CD56bight NK cells (FIG. 9A). These results indicate that PDGFRB has stronger promoter activity in CD56^{dim} NK cells than in CD56^{bright} NK cells, which may explain the differential regulation of PDGFRβ expression in the two NK cell populations (FIGS. 1H-1J).

### PDGFRβ Does Not Affect NK Cell Effector Functions

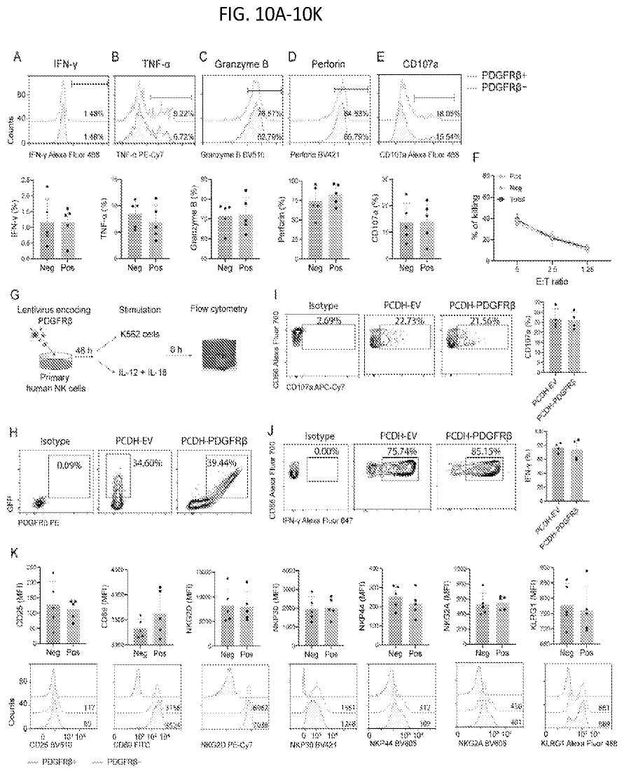

When we investigated the function of PDGFRβ in NK cells, we found that PDGFRβ⁺ and PDGFRβ⁻ NK cells had comparable IFN-γ, TNF-α, granzyme B, and perforin expression (FIGS. 10A-10D). The two populations also had similar CD107a expression levels and NK cell cytotoxicity against K562 cells (FIGS. 10E-10F), indicating that the presence of PDGFRβ is not essential for NK cell effector function. We also ectopically expressed PDGFRβ on primary NK cells, using a lentivirus that overexpressed PDGFRβ (FIGS. 10G-10H). The transduced cells showed similar expression levels of CD107a or IFN-γ compared to NK cells transduced with empty vector, in the stimulation of K562 cells or IL-12 plus IL-18, respectively (FIGS. 10I-10J). We also compared functional receptors on the surfaces of these two populations. PDGFRβ⁺ and PDGFRβ⁻ NK cells expressed similar levels of activation receptors (such as CD25, CD69, NKG2D, NKp30, and NKp44) and inhibitory receptors (such as NKG2A and KLRG1) (FIG. 10K).

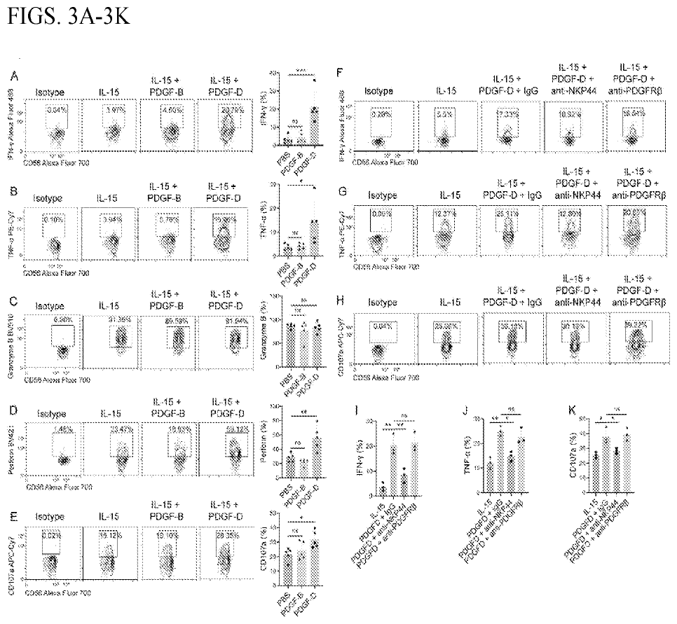

When PDGFRβ binds to its two ligands, PDGF-B or PDGF-D, it induces downstream signaling, including Ras/Raf/MAPK and PI3K/AKT, resulting in cell growth (5). We therefore treated IL-15-primed NK cells with PDGF-B or PDGF-D and then evaluated NK cell function. PDGF-D, but not PDGF-B, induced the expression of IFN-γ, TNF-α, perforin, and CD107a but not granzyme B (FIGS. 3A-3E). A prior study reported that PDGF-D can interact with NKp44 to stimulate the secretion of IFN-γ and TNF-α from NK cells (6). To determine whether that stimulation was dependent on NKp44 or PDGFRβ, we added NKp44- or PDGFRβ-neutralizing antibodies to the culture system. Blocking NKp44, but not PDGFRβ, significantly abrogated the increased expression of IFN-γ, TNF-α, and CD107a (FIGS. 3F-3K). These data indicate that PDGF-D-PDGFRβ signaling does not affect NK cell activation or effector functions.

### PDGFRβ Signaling Contributes to IL-15-Mediated NK Cell Survival

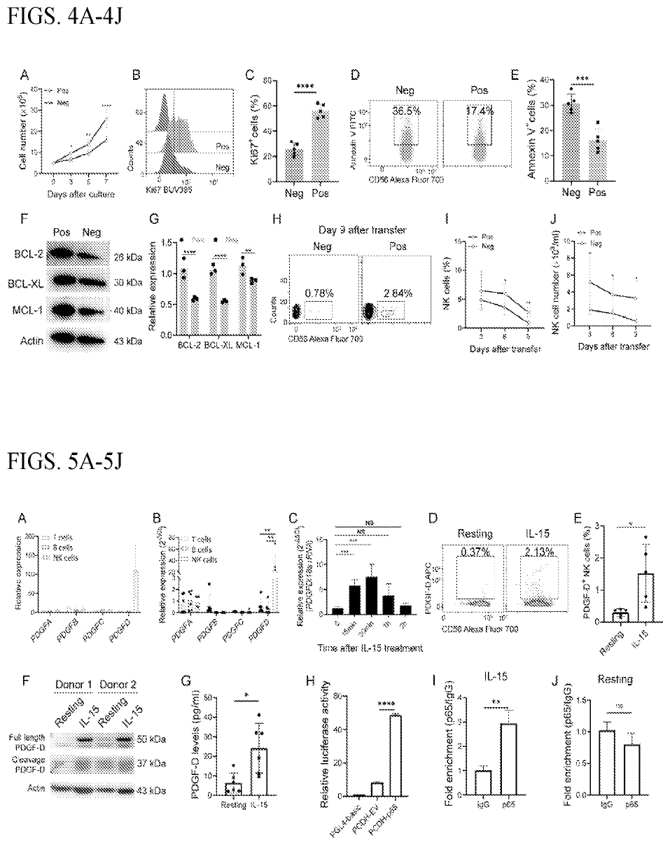

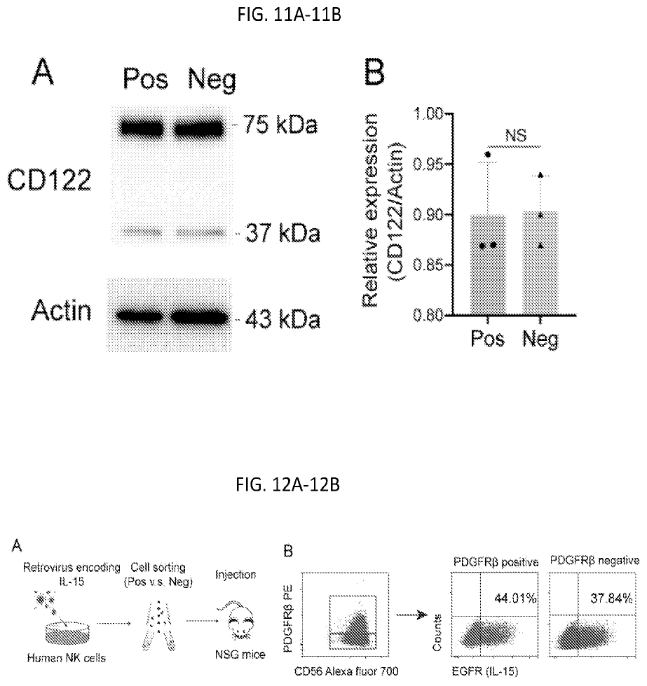

IL-15 is a key cytokine for NK cell proliferation and survival through pro-survival Bcl-2 family proteins, such as BCL-2, BCL-XL, and MCL-1 (11, 18-21). We therefore determined whether PDGFRβ on NK cells regulates IL-15-mediated cell proliferation or survival. PDGFRβ⁺ NK cells grew faster than PDGFRβ⁻ NK cells in vitro (FIG. 4A). PDGFRβ⁺ NK cells also showed significantly higher Ki67 expression compared with PDGFRβ⁻ NK cells (FIGS. 4B-4C). In addition, PDGFRβ⁺ NK cells had fewer annexin V⁺ apoptotic cells than PDGFRβ⁻ NK cells (FIGS. 4D-4E), indicating that PDGFRβ signaling enhances NK cell survival. Immunoblotting further demonstrated that PDGFRβ⁺ NK cells had significantly higher levels of BCL-2, BCL-XL, and MCL-1 compared to PDGFRβ⁻ NK cells (FIGS. 4F-4G). However, we found equivalent levels of the IL-15 receptor (CD122) in PDGFRβ⁺ and PDGFRβ⁻ NK cells (FIGS. 11A-11B), indicating that these two populations may have similar responsiveness to IL-15. To address whether PDGFRβ also promotes NK cell persistence in vivo, we stably transduced NK cells with soluble IL-15 (FIG. 12A). PDGFRβ⁺ and PDGFRβ⁻ NK cells were transduced with similar efficiency (FIG. 12B). The cells were then sorted, and equal numbers were injected into NSG mice (FIG. 12A). We found that PDGFRβ⁺ NK cells were persisting nine days after injection, while PDGFRβ⁻ NK cells could not (FIG. 4H). The percentage and the absolute number of PDGFRβ⁺ NK cells were also significantly higher than those of PDGFRβ⁻ NK cells 3 and 6 days after injection (FIGS. 4I-4J). Collectively, these results demonstrate the PDGFRβ contributes to IL-15-mediated NK cell survival in vitro and in vivo.

### IL-15 Maintains NK Cell Survival in Part Through a PDGF-D-PDGFRβ Autocrine Pathway

PDGFRβ needs to bind to its ligand to maintain NK cell survival, and our data showed that PDGFRβ⁺ NK cells grow better than PDGFRβ⁻ NK cells. We therefore hypothesized that NK cells might express ligands that recognize. Using BioGPS (http://biogps.org) to screen for genes that encode ligands of PDGFR, we found that human NK cells express high mRNA levels of PDGF-D but not PDGFA, PDGFB, or PDGFC (FIG. 5A). We confirmed this finding using qPCR, finding 20-fold-higher levels of mRNAs for PDGF-D in NK cells than for the other family members (FIG. 5B). Compared to T cells and B cells, only NK cells expressed high levels of PDGF-D (FIG. 5B). In addition, we found that IL-15 stimulation significantly increased mRNA and protein levels of PDGF-D in NK cells, as determined by qPCR (FIG. 5C), flow cytometry (FIGS. 5D-5E), immunoblotting (FIG. 5F), and enzyme-linked immunosorbent assay (FIG. 5G). Interestingly, we also detected a binding site for p65 in the promoter region of PDGF-D (FIG. 8C), and a luciferase reporter assay showed that p65 directly activated PDGF-D gene transcription (FIG. 5H). Moreover, ChIP-qPCR revealed that p65 was significantly enriched compared with a normal IgG control in IL-15-stimulated NK cells but not in resting NK cells (FIGS. 5I-5J). Taken together, our data demonstrate that NK cells express PDGF-D in an autocrine manner and that PDGF-D can be upregulated by p65 downstream of IL-15 signaling.

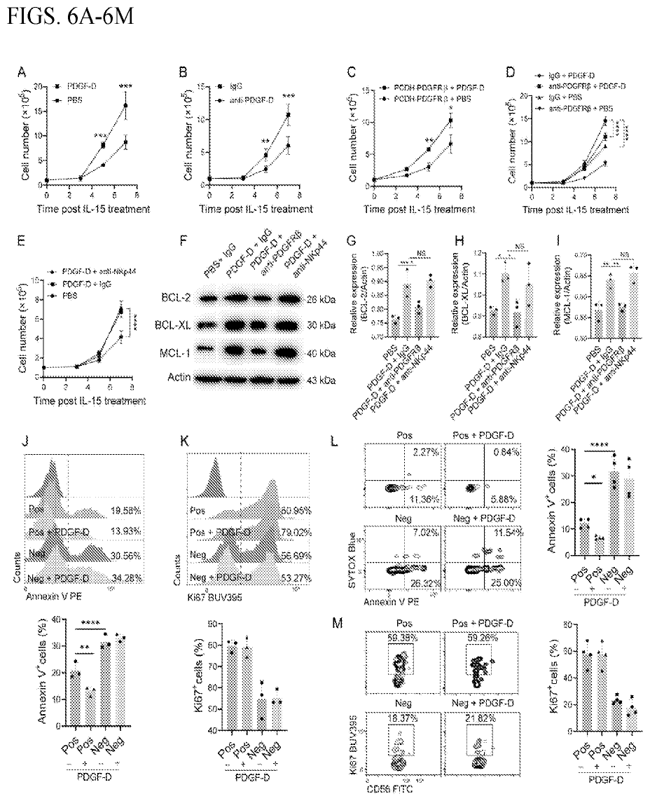

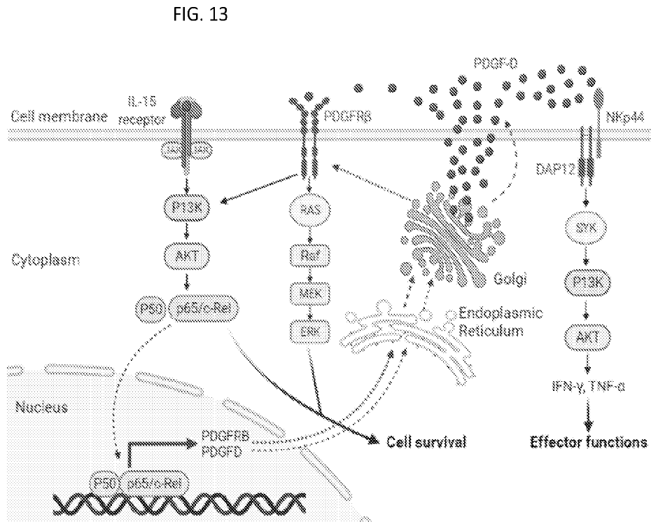

Finding that NK cells express PDGF-D led us to hypothesize that IL-15 may maintain NK cell survival through a PDGF-D-PDGFRβ autocrine pathway. PDGF-D treatment of NK cells promoted cell expansion in the presence of IL-15 (FIG. 6A). In contrast, PDGF-D-blocking antibody inhibited NK cell expansion (FIG. 6B). Also, PDGF-D enhanced the expansion of primary NK cells transduced with PDGFRβ (FIG. 6C). In addition, when we treated NK cells with a PDGFRβ-neutralizing antibody to block PDGF-D- PDGFRβ signaling, NK cell expansion triggered by PDGF-D decreased significantly (FIG. 6D). However, neutralizing NKp44 did not affect PDGF-D-mediated NK cell growth (FIG. 6E). An immunoblot showed that PDGF-D treatment increased the expression levels of BCL-2, BCL-XL, and MCL-1, in an NKp44-independent but PDGFRβ-dependent manner (FIGS. 6F-6I). Further studies showed that PDGF-D inhibited apoptosis of PDGFRβ⁺ NK cells, but not PDGFRβ⁻ NK cells, in vitro and in vivo (FIGS. 6J-6L). PDGF-D did not affect NK cell proliferation as shown by similar levels of Ki67 in vitro and in vivo (FIGS. 6K-6M), suggesting that PDGF-D promotes IL-15-mediated NK cell survival but not proliferation, though PDGFRβ⁺ NK cells are more proliferative than PDGFRβ⁻ NK cells (FIGS. 4B, 4C, 6K). Thus, our findings reveal a novel mechanism by which IL-15 promotes NK cell survival: IL-15 induces NK cells to express both PDGF-D and PDGFRβ; then engagement of PDGFRβ by PDGF-D promotes NK cell survival (FIG. 13).

### Discussion

In this study, we showed that PDGF-D-PDGFRβ signaling, a potent stimulator of cell growth and motility, activates an autocrine pathway that contributes to IL-15-mediated survival of human NK cells. Our findings therefore expand our understanding of the mechanism by which IL-15 signaling regulates NK cell immunity. Moreover, introducing PDGF-D-PDGFRβ signaling into NK cells might help enhance their survival and improve NK cell-based immunotherapy.

NK cells in humans and mice share many features, including expression of the transcription factors T-bet and Eomes and activation-induced production of IFN-γ, TNF-α, granzyme B, and perforin. In humans, NK cells are typically defined as CD3⁻CD56⁺ cells, and they can be further divided into CD3⁻CD56^{dim} and CD3⁻CD56^{bright} populations (1, 22). In mice, NK cells are defined as CD3⁻NK1.1⁺ cells that typically express NKp46, CD49b, CD11b, CD27 but not CD127 (23). In this study, we found that IL-15 could not induce PDGFRβ expression in mouse NK cells, indicating that PDGF-D-PDGFRβ signaling does not contribute to IL-15-mediated cell proliferation and survival in that system. We also found that only CD56^{dim} NK cells expressed PDGFRβ after IL-15 stimulation. CD56^{dim} NK cells, which account for more than 90% of peripheral NK cells, are cytolytic, whereas the CD56^{bright} subset is immunoregulatory, mainly through cytokine production (22). Because CD56^{bright} cells are immature precursors of mature CD56^{dim} NK cells (9, 22, 24), our findings indicate that only mature NK cells can express PDGFRβ. This is in agreement with a previous report showing that both CD56^{bright} and CD56d 1 m NK cells robustly express IL-15Rβ/γ, while these two NK cell subsets may have separate IL-15-mediated signaling pathways that activate different transcriptional regulatory networks (25). Gene expression levels should also be controlled by chromatin accessibility (14). Our analysis of publicly available databases indeed indicated that, in the promoter region of the PDGFRB locus, CD56^{dim} NK cells are more accessible to transposons with higher levels of the H3K27Ac histone modification than are CD56^{bright} NK cells.

PDGF-D-PDGFRβ signaling has important functions in the regulation of cell growth and survival. PDGF-D has been recognized as a ligand of NKp44, one of the natural cytotoxicity receptors expressed by activated NK cells (6). PDGF-D prompted NK cells to secrete IFN-γ and TNF-α, arresting the growth of tumor cells (6). We confirmed that PDGF-D induced the production of IFN-γ, TNF-α, and granzyme B in IL-15-activated NK cells, and that induction was dependent on NKp44 but not on PDGFRβ. In addition, we provided direct evidence that. Therefore, our findings, together with the prior report, suggest that PDGF-D not only enhances effector function through NKp44 but also promotes cell survival through PDGFRβ in human NK cells. In cancer, PDGF-D is abundant and is known to stimulate tumor growth and angiogenesis through PDGFRβ (6, 26-28), Therefore, future development of PDGF-D or PDGFRβ inhibitors to target tumor cells for cancer therapy should be pursued with caution, as inhibiting PDGF signaling might impair host anti-tumor responses by NK cells. On the other hand, selectively introducing PDGF signaling into NK cells might benefit NK cell expansion, persistence, and enhancement of effector function during NK cell-based immunotherapy.

Large granular lymphocyte (LGL) leukemia is a lymphoproliferative disease characterized by a clonal expansion of cytotoxic T or NK cells. Aggressive T-cell and NK-cell LGL leukemia is resistant to therapy, producing a poor prognosis (29). It is recognized that IL-15 and PDGF play crucial roles in LGL leukemia expansion by promoting NK-cell or leukemic LGL survival (8, 30). LGL leukemia cells promote their survival by expressing high levels of PDGFRβ and PDGF-B to activate an autocrine regulatory pathway (8). Therefore. PDGFRβ signaling not only contributes to normal NK cell survival but also is a key survival factor in LGL leukemia. However, the factors that prompt LGLs to express PDGF remain to be discovered (8). Our findings indicate that IL-15 signaling is a key initiation factor that drives PDGF-D and PDGFRβ expression in normal NK cells. We reported earlier that IL-15 plays a central role in the genesis of LGL leukemia and that overexpression of IL-15 as a single growth factor can initiate the leukemic transformation of LGLs (12, 31, 32). We therefore hypothesize that IL-15-PDGF signaling may play a causal role in the pathogenesis of LGL leukemia and that targeting IL-15-PDGF signaling might be a potential therapy for the disease.

In conclusion, we report a previously unknown role for PDGF-D-PDGFRβ signaling: positive regulation of IL-15-mediated cell survival of human NK cells without an effect on NK cell effector functions. Thus, our findings advance the mechanistic understanding of IL-15 signaling in NK cell immunity and point to the introduction of PDGF signaling into NK cells as a promising strategy for advancing NK cell-based therapies against tumors with the precaution of leukemogenesis potentially driven by the signaling.

### Materials and Methods

### Isolation of Primary Human NK Cells

Peripheral blood samples from de-identified healthy donors were obtained from the Michael Amini Transfusion Medicine Center of City of Hope National Medical Center under institutional review board-approved protocols. NK cells were enriched using the RosetteSep Human NK Cell Enrichment Cocktail (STEMCELL Technologies) and Ficoll-Paque (GE Healthcare). The purity of primary NK cells (CD3⁻CD56⁺) was confirmed with flow cytometry. CD56^{bright} and CD56^{dim} NK cells were sorted with a FACSAria Fusion Flow Cytometer (BD Biosciences).

### Antibodies, Cytokines, and Inhibitors

Fluorochrome-conjugated mouse anti-human antibodies against CD3 (UCHT1), CD56 (B159), PDGFRα (αR1), PDGFRβ (28D4), IFN-γ (4S. B3), TNF-α (MAb11), CD107a (H4A3), granzyme B (GB11), perforin (δG9), CD25 (M-A251), CD69 (FN50), NKG2D (1D11), NKp30 (p30-15), NKp44 (p44-8), NKG2A (131411), and Ki67 (B56) and isotype controls were purchased from BD Biosciences. Anti-human KLRG1 (13F12F2) was purchased from eBioscience. APC-conjugated human PDGF-D antibody was purchased from Assaypro LLC. Anti-mouse CD3 (17A2), NK1.1 (PK136), and PDGFRβ (APBS) were purchased from BioLegend. PDGF receptor β (28E1) rabbit mAb (#3169), Phospho-NF-κB p65 (Ser536) (93H1) rabbit mAb (#3033), NF-κB p65 (D14E12) rabbit mAb (#8242), Bcl-2 (124) mouse mAb (#15071), Bcl-xL (54H6) rabbit mAb (#2764), Mcl-1 (D2W9E) rabbit mAb (#94296), β-tubulin (9F3) rabbit mAb (#2128), and lamin B1 (D9V6H) rabbit mAb (#13435) were purchased from Cell Signaling Technology. Alexa Fluor 488-conjugated anti-PDGF receptor beta (sc-19995 AF488) was purchased from Santa Cruz. Recombinant anti-SCDGFB/PDGF-D antibody (ab181845), recombinant anti-IL2 receptor beta/p75 antibody (ab271040), and recombinant anti-sodium-potassium ATPase antibody (ab76020) were purchased from Abcam. Beta-actin monoclonal antibody (66009-1-Ig) was purchased from Proteintech. Recombinant human IL-2 (200-02), IL-12 p70 (200-12), and IL-15 (200-15); recombinant murine IL-2 (402-ML-020), IL-12 (419-ML-010/CF), and IL-15 (447-ML-010/CF); and recombinant human IL-18 (B001-5), PDGF-BB (220-BB-010), and PDGF-DD (1159-SB-025) were purchased from R&D Systems. Purified anti-human CD336 (NKp44) antibody (325104), purified mouse IgG1, and x isotype control antibody (40140150) were purchased from BioLegend. Human PDGFR beta antibody (AF385) was purchased from R&D Systems. Wortmannin, afuresertib, TPCA-1, decernotinib, AZD6244, CI-1040, and cycloheximide were purchased from Selleck Chemicals. Rapamycin, Torin1, and STAT5-IN-1 were purchased from MedChemExpress. Actinomycin D (Cat. A9415) was purchased from Sigma-Aldrich. Brefeldin A was purchased from BioLegend.

### NK Cell Culture, Transduction, and Treatment

NK cells were cultured in RPMI 1640 with 10% heat-inactivated FBS (Gibco) at 37° C. in a 5% CO₂ humidified incubator. For NK cell transduction, PDGFRB cDNA ORF clone (Cat: HG10514-G) was purchased from Sino Biological and cloned into pCDH-CMV-MCS-EF1-copGFP lentivirus vector (System Biosciences). To produce lentivirus, we transfected the lentiviral transfer vector DNA, together with psPAX2 packaging (Addgene) and pMD2.G envelope plasmid DNA (Addgene), into HEK293T cells; using a polyethyenimine (PEI) transfection protocol (Polysciences). Concentrated lentivirus was added to primary NK cells cultured in RPMI 1640 medium supplemented with 10% FCS and 10 jig/mL polybrene with 2,000×g centrifugation for 2 h. Cells were cultured for an additional 48 h. For cytokine treatment, NK cells were treated with IL-2, IL-12, IL-15, IL-18, or a combination for the indicated time. The cells were then collected for flow cytometry analysis. For the inhibition assay, NK cells were pretreated with the indicated inhibitors for 1 h, washed twice with RPMI 1640. and then treated with IL-15 for 24 h.

### Mouse NK Cell Isolation and Treatment

Mouse NK cells were isolated from the spleen of C57BL/6J or IL-15 transgenic mice, using the EasySep Mouse NK Cell Isolation Kit (STEMCELL Technologies) as previously described (11). Cells were treated with IL-2, IL-12, IL-15, or IL-12 plus IL-15 for 24 h and then collected for flow cytometry.

### Flow Cytometry

Cells were stained with the indicated cell-surface markers and/or fixed/permeabilized using a Fixation/Permeabilization Kit (eBioscience). For intracellular staining of IFN-γ and PDGF-D, 5 μg/ml Brefeldin A (BioLegend) was added for 4 h before cell harvest. Intracellular staining of Ki67 was performed by fixing and permeabilizing with the Foxp3/Transcription Factor Staining Kit (eBioscience). For CD107a staining, CD107a antibody was added into the culture for 4 h in the presence of Brefeldin A. Annexin V staining was performed using an FITC Annexin V Apoptosis Detection Kit according to the manufacturer's protocol (BD Biosciences). Flow cytometry analysis was performed on BD LSRFortessa X-20 (BD Biosciences). Data were analyzed using NovoExpress software (Agilent Technologies).

### Immunofluorescence Assay

Resting NK cells and IL-15-treated NK cells were fixed with 4% formaldehyde, blocked with 5% BSA, and then stained with Alexa Fluor 488-conjugated anti-PDGF receptor beta and anti-sodium-potassium ATPase antibody overnight at 4° C. The cells were washed and incubated with Alexa Fluor 647-conjugated goat anti-rabbit secondary antibody (Jackson ImmunoResearch) at room temperature for 1 h. Cells were rinsed three times in 1×PBS and 1 drop of the Diamond Antifade Mountant with DAPI (Thermo Scientific) was then applied. Cover slide-mounted specimens were visualized, and images were acquired using a Zeiss microscope.

Quantitative Real-Time RT-PCR (qPCR) and Immunoblotting

RNA was isolated using an RNeasy Mini Kit (QIAGEN) and reversely transcribed to cDNA with a PrimeScript RT Reagent Kit with gDNA Eraser (Takara Bio) according to the manufacturer's protocol. mRNA expression levels were analyzed using SYBR Green qPCR Master Mix and a QuantStudio 12K Flex Real-Time PCR System (both from Thermo Fisher Scientific). Primer sequences used are as follows: PDGFRB forward, 5′-TGATGCCGAGGAACTATTCATCT-3′ (SEQ ID NO:11); PDGFRB reverse, 5′-TTTCTTCTCGTGCAGTGTCAC-3′ (SEQ ID NO:12); PDGF-D forward, 5′-TTGTACCGAAGAGATGAGACCA-3′ (SEQ ID NO:13); PDGF-D reverse, 5′-GCTGTATCCGTGTATTCTCCTGA-3′ (SEQ ID NO:14); 18S rRNA forward, 5′-TGTGCCGCTAGAGGTGAAATT-3′ (SEQ ID NO:15); and 18S rRNA reverse, 5′-TGGCAAATGCTTTCGCTTT-3′ (SEQ ID NO:16). Relative amplification values were normalized to the amplification of 18S rRNA. Immunoblotting was performed according to standard procedures, as previously described (11). Cellular fractionation separation was performed using NE-PER Nuclear and Cytoplasmic Extraction Reagents and the Mem-PER Plus Membrane Protein Extraction Kit (Thermo Scientific).

### Enzyme-Linked Immunosorbent Assay (ELISA)

Primary NK cells (5×10⁶/well) were plated in a 6-well plate in RPMI 1640 supplemented with 10% FBS. The cells were treated with recombinant human IL-15 (50 ng/ml) for 24 h, and supernatants were collected and frozen at −80° C. for later use. The PDGF-D concentration in the supernatant was measured using a Human PDGF-DD Quantikine ELISA Kit (DDDOO, R&D Systems).

### Luciferase Reporter Assay

The promoter regions of PDGFRB and PDGF-D were amplified and cloned into the pGL4-basic luciferase reporter vector (Promega). HEK293T cells purchased from ATCC were co-transfected with the pGL4-PDGFRB or pGL4-PDGF-D reporter plasmid as well as with a p65-overexpression plasmid or empty vector. A pRL-TK Renilla reporter plasmid (Promega) was added to normalize transfection efficiency. The cells were harvested for lysis 24 h after transfection, and luciferase activity was quantified fluorimetrically with Dual-Luciferase Reporter Assay System (Promega). A p65 overexpression plasmid was used as previously described (33). Primer sequences for cloning the PDGFRB and PDGF-D promoters were as follows: PDGFRB forward, 5′-CGGGGTACCCAAAGACCTGGCCAGGCCCCCTCT-3′ (SEQ ID NO:17); PDGFRB reverse, 5′-CCGCTCGAGCTGGCAGCCTCAGGAGCTCACACCA-3′ (SEQ ID NO:18); PDGF-D forward, 5′-CCGCTCGAGCAAAGAGATTAGGAACTTTATTTCT-3′ (SEQ ID NO:19); and PDGF-D reverse, 5′-CCCAAGCTTTGACGGGACAAACAACAGGTTGA-3′ (SEQ ID NO:20).

### Chromatin Immunoprecipitation (ChIP) Assay

Primary NK cells were treated with IL-15 for 1 h. The cells were cross-linked in 1% formaldehyde and quenched with glycine buffer. ChIP assays were carried out using a Pierce Magnetic ChIP Kit (Cat No.26157, Thermo Scientific) according to the manufacturer's instructions. Digested chromatin was incubated overnight with a p65 ChIP-grade antibody (#8242, Cell Signaling Technology) or IgG control antibody (#3900, Cell Signaling Technology). The enriched chromatin was analyzed by qPCR using the following primers:

PDGFRB forward,
(SEQ ID NO: 21)
5′-AAATGATCTCCCTGGGTGCCA-3′;
PDGFRB reverse,
(SEQ ID NO: 22)
5′-CGCGTGCGTCTGTTTTCAA-3′;
PDGF-D forward,
(SEQ ID NO: 23)
5′-TCCTTAGTGTCTCTCCCAGGG-3′;
and
PDGF-D reverse,
(SEQ ID NO: 24)
5′-AAATTTAGGTTTGTGGGCCATG-3′.

### ⁵¹Cr Release Cytotoxicity Assay

NK cell cytotoxicity against K562 cells was evaluated by standard ⁵¹Cr release assays as previously described (11). PDGFRβ⁺ and PDGFRβ⁻ NK cells were sorted and co-cultured with ⁵¹Cr-labeled K562 cells in a 96-well V-bottom plate at ratios of 5:1, 2.5:1, and 1.25:1 for 4 h at 37° C. in a 5% CO2 incubator. Supernatant harvested from each well was transferred into a 96-well Luma plate and analyzed using a MicroBeta Scintillation Counter (Wallac, PerkinElmer). Percentages of killing were calculated using the following equation:

$$\% specific lysis = 100 \times ( ( test {}^{51} Cr release ) - ( spontaneous {}^{51} Cr release ) ) / ( ( maximal {}^{51} Cr release ) - ( spontaneous {}^{51} Cr release ) ) .$$

### NK Cell Transduction and Adoptive Transfer

The full-length human IL-15 sequence was cloned into a pCIR retrovirus vector. To produce retrovirus, GP2-293 cells were transfected with that vector, using Lipofectamine 3000 reagent (Thermo Fisher). NK cell transduction was performed using RetroNectin (Takara Bio)-coated plates with 2,000×g centrifugation for 2 h, as described previously (3). The infected cells were washed and cultured with rhlL-2 (1,000 IU/ml) for 48 h. Transduced NK cells were expanded for 7 days, using irradiated (25 Gy) autologous PBMCs as described previously (3). 1×10⁷ sorted PDGFRβ⁺ or PDGFRβ⁻ NK cells were injected into NOD/SCID/IL-2rg (NSG) mice (The Jackson Laboratory), followed by detection of survival of PDGFRβ⁺ or PDGFRβ⁻ NK cells in peripheral blood by flow cytometry on days 3, 6, and 9after adoptive transfer. To investigate the effect of PDGF-D in this setting, 5×10⁶ sorted PDGFRβ⁻ or PDGFRβ⁻ NK cells were intravenously co-injected with PDGF-D (1 μg per mouse) or phosphate-buffered saline on day 0 and PDGF-D were administered similarly on both day 1 and 2. On day 3, mice were sacrificed and peripheral blood samples were collected to analyze survival of PDGFRβ⁺ or PDGFRβ⁻ NK cells.

### Statistical Analysis

Unpaired Student's t-tests (two-tailed) were performed using Prism software. One-way or two-way ANOVA was performed when three or more independent groups were compared. P values were adjusted for multiple comparisons using Holm-Sidak's procedure. A P value<0.05 was considered statistically significant.

INFORMAL SEQUENCE LISTING

SEQ ID NO: 1 Nucleic acid encoding IL-2 signal peptide
ATGTACAGGATGCAACTCCTGTCTTGCATTGCACTAAGTCTTGCACTTGTCACAA
ACAGT
SEQ ID NO: 1 Nucleic acid encoding IL-15 protein
GGCATTCATGTCTTCATTTTGGGCTGTTTCAGTGCAGGGCTTCCTAAAACAGAAG
CCAACTGGGTGAATGTAATCAGCGACCTCAAGAAGATCGAGGACTTGATCCAGT
CCATGCACATAGATGCTACTTTATATACGGAAAGTGATGTTCACCCCAGTTGCAA
AGTAACAGCAATGAAGTGCTTTCTCTTGGAGTTACAAGTTATTTCACTTGAGTCC
GGAGATGCAAGTATTCATGATACAGTAGAAAATCTGATCATCCTAGCAAACAAC
AGTTTGTCTTCTAATGGGAATGTAACAGAATCTGGATGCAAAGAATGTGAGGAA
CTGGAGGAAAAAAATATTAAAGAATTTTTGCAGAGTTTTGTACATATTGTCCAAA
TGTTCATCAACACTTCT
SEQ ID NO: 3 Nucleic acid encoding soluble IL-15
ATGTACAGGATGCAACTCCTGTCTTGCATTGCACTAAGTCTTGCACTTGTCACAA
ACAGTGGCATTCATGTCTTCATTTTGGGCTGTTTCAGTGCAGGGCTTCCTAAAAC
AGAAGCCAACTGGGTGAATGTAATCAGCGACCTCAAGAAGATCGAGGACTTGAT
CCAGTCCATGCACATAGATGCTACTTTATATACGGAAAGTGATGTTCACCCCAGT
TGCAAAGTAACAGCAATGAAGTGCTTTCTCTTGGAGTTACAAGTTATTTCACTTG
AGTCCGGAGATGCAAGTATTCATGATACAGTAGAAAATCTGATCATCCTAGCAA
ACAACAGTTTGTCTTCTAATGGGAATGTAACAGAATCTGGATGCAAAGAATGTG
AGGAACTGGAGGAAAAAAATATTAAAGAATTTTTGCAGAGTTTTGTACATATTGT
CCAAATGTTCATCAACACTTCT
SEQ ID NO: 4 IL-2 signal peptide:
MYRMQLLSCIALSLALVINS
SEQ ID NO: 5 IL-15 protein
GIHVFILGCFSAGLPKTEANWVNVISDLKKIEDLIQSMHIDATLYTESDVHPSCKVTA
MKCFLLELQVISLESGDASIHDTVENLIILANNSLSSNGNVTESGCKECEELEEKNIKE
FLQSFVHIVQMFINTS
SEQ ID NO: 6 soluble IL-15
MYRMQLLSCIALSLALVTNSGIHVFILGCFSAGLPKTEANWVNVISDLKKIEDLIQSM
HIDATLYTESDVHPSCKVTAMKCFLLELQVISLESGDASIHDTVENLIILANNSLSSNG
NVTESGCKECEELEEKNIKEFLQSFVHIVQMFINTS
SEQ ID NO: 7 PDGF-D
MHRLIFVYTLICANFCSCRDTSATPQSASIKALRNANLRRDESNHLTDLYRRDETIQV
KGNGYVQSPRFPNSYPRNLLLTWRLHSQENTRIQLVFDNQFGLEEAENDICRYDFVE
VEDISETSTIIRGRWCGHKEVPPRIKSRTNQIKITFKSDDYFVAKPGFKIYYSLLEDFQP
AAASETNWESVTSSISGVSYNSPSVTDPTLIADALDKKIAEFDTVEDLLKYFNPESWQ
EDLENMYLDTPRYRGRSYHDRKSKVDLDRLNDDAKRYSCTPRNYSVNIREELKLAN
VVFFPRCLLVQRCGGNCGCGTVNWRSCTCNSGKTVKKYHEVLQFEPGHIKRRGRAK
TMALVDIQLDHHERCDCICSSRPPR
SEQ ID NO: 8-PDGFRβ protein
MRLPGAMPALALKGELLLLSLLLLLEPQISQGLVVTPPGPELVLNVSSTFVLTCSGSA
PVVWERMSQEPPQEMAKAQDGTFSSVLTLTNLTGLDTGEYFCTHNDSRGLETDERK
RLYIFVPDPTVGFLPNDAEELFIFLTEITEITIPCRVTDPQLVVTLHEKKGDVALPVPYD
HQRGFSGIFEDRSYICKTTIGDREVDSDAYYVYRLQVSSINVSVNAVQTVVRQGENIT
LMCIVIGNEVVNFEWTYPRKESGRLVEPVTDFLLDMPYHIRSILHIPSAELEDSGTYTC
NVTESVNDHQDEKAINITVVESGYVRLLGEVGTLQFAELHRSRTLQVVFEAYPPPTV
LWFKDNRTLGDSSAGEIALSTRNVSETRYVSELTLVRVKVAEAGHYTMRAFHEDAE
VQLSFQLQINVPVRVLELSESHPDSGEQTVRCRGRGMPQPNIIWSACRDLKRCPRELP
PTLLGNSSEEESQLETNVTYWEEEQEFEVVSTLRLQHVDRPLSVRCTLRNAVGQDTQ
EVIVVPHSLPFKVVVISAILALVVLTIISLIILIMLWQKKPRYEIRWKVIESVSSDGHEYI
YVDPMQLPYDSTWELPRDQLVLGRTLGSGAFGQVVEATAHGLSHSQATMKVAVK
MLKSTARSSEKQALMSELKIMSHLGPHLNVVNLLGACTKGGPIYIITEYCRYGDLVD
YLHRNKHTFLQHHSDKRRPPSAELYSNALPVGLPLPSHVSLTGESDGGYMDMSKDE
SVDYVPMLDMKGDVKYADIESSNYMAPYDNYVPSAPERTCRATLINESPVLSYMDL
VGFSYQVANGMEFLASKNCVHRDLAARNVLICEGKLVKICDFGLARDIMRDSNYIS
KGSTFLPLKWMAPESIFNSLYTTLSDVWSFGILLWEIFTLGGTPYPELPMNEQFYNAI
KRGYRMAQPAHASDEIYEIMQKCWEEKFEIRPPFSQLVLLLERLLGEGYKKKYQQV
DEEFLRSDHPAILRSQARLPGFHGLRSPLDTSSVLYTAVQPNEGDNDYIIPLPDPKPEV
ADEGPLEGSPSLASSTLNEVNTSSTISCDSPLEPQDEPEPEPQLELQVEPEPELEQLPDS
GCPAPRAEAEDSFL
SEQ ID NO: 9-PDGFRβ protein
MSQEPPQEMAKAQDGTFSSVLTLTNLTGLDTGEYFCTHNDSRGLETDERKRLYIFVP
DPTVGFLPNDAEELFIFLTEITEITIPCRVTDPQLVVTLHEKKGDVALPVPYDHQRGFS
GIFEDRSYICKTTIGDREVDSDAY
SEQ ID NO: 10-PDGFRβ protein
MRLPGAMPALALKGELLLLSLLLLLEPQISQGLVVTPPGPELVLNVSSTFVLTCSGSA
PVVWERMSQEPPQEMAKAQDGTFSSVLTLTNLTGLDTGEYFCTHNDSRGLETDERK
RLYIFVPDPTVGFLPNDAEELFIFLTEITEITIPCRVTDPQLVVTLHEKKGDVALPVPYD

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

### REFERENCES

1. Freud et al, Immunity 47, 820-833 (2017).
2. Yilmaz et al, J Hematol Oncol 13, 168 (2020).
3. Ma et al., Cancer Res 81, 3635-3648 (2021).
4. Liu et al., N Engl J Med 382, 545-553 (2020).
5. Andrae et al, Genes Dev 22, 1276-1312 (2008).
6. Barrow et al., Cell 172, 534-548 e519 (2018).
7. Gersuk et al, Nat Immun Cell Growth Regul 5, 283-293 (1986).
8. Yang et al., Blood 115, 51-60 (2010).
9. Yu et al., Blood 115, 274-281 (2010).
10. Cooper et al., Blood 97, 3146-3151 (2001).
11. Ma et al, J Exp Med 218 (2021).
12. Yu et al., J Clin Invest 121, 1456-1470 (2011).
13. Mishra, et al, Clin Cancer Res 20, 2044-2050 (2014).
14. Klemm et al, Nat Rev Genet 20, 207-220 (2019).
15. Karmodiya et al, BMC Genomics 13, 424 (2012).
16. Kouzarides et al, Cell 128, 693-705 (2007).
17. Collins et al., Cell 176, 348-360 e312 (2019).
18. Wang et al., Nat Immunol 20, 10-17 (2019).
19. Carson et al., J Exp Med 180, 1395-1403 (1994).
20. Huntington et al., Nat Immunol 8, 856-863 (2007).
21. Carson et al., J Clin Invest 99, 937-943 (1997).
22. Freud et al, Semin Immunol 26, 132-137 (2014).
23. Yu et al, Trends Immunol 34, 573-582 (2013).
24. Luetke-Eversloh et al, Front Immunol 4, 499 (2013).
25. Wagner et al., J Clin Invest 127, 4042-4058 (2017).
26. Liu et al., Clin Cancer Res 17, 3638-3648 (2011).
27. Xu et al, Cancer Res 65, 5711-5719 (2005).
28. LaRochelle et al., Cancer Res 62, 2468-2473 (2002).
29. Moignet et al, Am Soc Clin Oncol Educ Book 38, 616-625 (2018).
30. Zhang et al., PNAS USA 105, 16308-16313 (2008).
31. Mishra et al., Cancer Cell 22, 645-655 (2012).
32. Fehniger et al., J Exp Med 193, 219-231 (2001).
33. Dong et al., Cancer Discov 9, 1422-1437 (2019).

What is claimed is:

1. A genetically-modified NK cell that expresses:
(i) platelet-derived growth factor (PDGF);
(ii) platelet-derived growth factor receptor (PDGFR); or
(iii) PDGF and PDGFR.

2. The genetically-modified NK cell of claim 1, wherein the PDGF is platelet-derived growth factor D (PDGF-D) and wherein the PDGFR is platelet-derived growth factor receptor beta (PDGFRβ).

3. The genetically-modified NK cell of claim 1 that expresses PDGF.

4. The genetically-modified NK cell of claim 1 that expresses PDGFR.

5. The genetically-modified NK cell of claim 1 that expresses PDGF and PDGFR.

6. The genetically-modified NK cell of claim 1, wherein the genetically-modified NK cell expresses an increased level of PDGF relative to a non-genetically-modified NK cell.

7. The genetically-modified NK cell of claim 1, wherein the genetically-modified NK cell expresses an increased level of PDGFR relative to a non-genetically-modified NK cell.

8. The genetically-modified NK cell of claim 1, wherein the genetically-modified NK cell comprises an exogenous nucleic acid that expresses PDGF.

9. The genetically-modified NK cell of claim 1, wherein the genetically-modified NK cell comprises an exogenous nucleic acid that expresses PDGFR.

10. The genetically-modified NK cell of claim 9, wherein the exogenous nucleic acid is under inducible control.

11. The genetically-modified NK cell of claim 1, wherein the genetically-modified NK cell constitutively expresses PDGF and/or PDGFR.

12. The genetically-modified NK cell of claim 1, wherein the genetically-modified NK cell is a CD56^{dim} NK cell.

13. The genetically-modified NK cell of claim 1, wherein the genetically-modified NK cell is a human NK cell.

14. The genetically-modified NK cell of claim 1, wherein the genetically-modified NK cell expresses PD-L1.

15. The genetically-modified NK cell of claim 14, wherein the genetically-modified NK cell comprises an exogenous nucleic acid that expresses PD-L1.

16. The genetically-modified NK cell of claim 15, wherein the genetically-modified NK cell constitutively expresses PD-L1.

17. The genetically-modified NK cell of claim 1, wherein the genetically-modified NK cell expresses IL-15.

18. The genetically-modified NK cell of claim 17, wherein the genetically-modified NK cell comprises an exogenous nucleic acid that expresses IL-15.

19. The genetically-modified NK cell of claim 18, wherein the genetically-modified NK cell constitutively expresses IL-15.

20. The genetically-modified NK cell of claim 17, wherein the IL-15 is soluble IL-15.

21. The NK cell of claim 1, wherein the genetically-modified NK cell expresses truncated epidermal growth factor receptor (tEGFR) protein.

22. The genetically-modified NK cell of claim 22, wherein the genetically-modified NK cell comprises an exogenous nucleic acid that expresses tEGFR protein.

23. The genetically-modified NK cell of claim 22, wherein the genetically-modified NK cell constitutively expresses tEGFR protein.

24. The genetically-modified NK cell of claim 1, wherein the genetically-modified NK cell comprises an exogenous nucleic acid that expresses a chimeric antigen receptor.

25. The genetically-modified NK cell of claim 1, wherein the genetically-modified NK cell is an activated cord blood NK cell.

26. The NK cell of any one of claim 1, wherein the genetically-modified NK cell is derived from umbilical cord blood NK cells.

27. The NK cell of claim 26, wherein the umbilical cord blood NK cells were incubated with IL-12, IL-15, IL-18, or a combination of two or more thereof.

28. The NK cell of claim 1, wherein the genetically-modified NK cell comprises a chimeric antigen receptor (CAR).

29, The NK cell of claim 1, wherein the genetically-modified NK cell does not comprise a CD19 chimeric antigen receptor (CAR).

30. A pharmaceutical composition comprising the genetically-modified NK cell of claim 1.

31. A population of genetically-modified NK cells comprising a plurality of the genetically-modified NK cell of claim 1.

32. A pharmaceutical composition comprising the population of natural killer cells of claim 31.

33. A method of treating cancer in a subject in need thereof, the method comprising administering to the subject an effective amount of a population of the genetically-modified NK cell of claim 1.

34. The method of claim 33, wherein the cancer is lung cancer, neuroblastoma, glioma, myelodysplastic syndrome, leukemia, lymphoma, liver cancer, prostate cancer, pancreatic cancer, gastric cancer, head and neck cancer, multiple myeloma, biliary tract cancer, ovarian cancer, melanoma, or colorectal cancer. cancer.

35. The method of claim 34, wherein the lung cancer is non-small cell lung

36. The method of claim 34, wherein the cancer is leukemia.

37. The method of claim 36, wherein the leukemia is acute myeloid leukemia, chronic myeloid leukemia, or lymphoblastic leukemia.

38. The method of claim 34, wherein the cancer is lymphoma.

39. The method of claim 38, wherein the lymphoma is B cell lymphoma or non-Hodgkin lymphoma.

40. The method of claim 33, wherein the patient is refractory to chemotherapy.

41. The method of claim 33, wherein the patient is refractory to a PD-1 inhibitor and/or a PD-L1 inhibitor.

42. The method of claim 33, further comprising administering to the patient an effective amount of a checkpoint inhibitor

43. The method of claim 42, wherein the checkpoint inhibitor is a PD-1 inhibitor.

44. The method of claim 43, wherein the PD-1 inhibitor is pembrolizumab, nivolumab, cemiplimab, dostarlimab, JTX-4014, spartalizumab, camrelizumab, sintilimab, tislelizumab, toripalimab, retifanlimab, AMP-224, or AMP-514.

45. The method of claim 42, wherein the checkpoint inhibitor is a PD-L1 inhibitor.

46. The method of claim 45, wherein the PD-L1 inhibitor is atezolizumab, avelumab, durvalumab, envafolimab, cosibelimab, AUNP12, CA-170, or BMS-986189.

47. The method of claim 42, wherein the checkpoint inhibitor is a CTLA-4inhibitor.

48. The method of claim 47, wherein the CTLA-4 inhibitor is ipilimumab.

49. A method of expanding a population of natural killer cells in vitro, the method comprising contacting the NK cells with an effective amount of platelet-derived growth factor D (PDGF-D).

50. The method of claim 49, further comprising contacting the NK cells with an effective amount of IL-2, IL-12, IL-15, IL-18, or a combination of two or more thereof.

51. The method of claim 49, further comprising incubating the NK cells with K562 feeder cells.

52. The method of claim 51, wherein the K562 feeder cells express membrane-bound IL-21 and CD-137L and exogenous IL-2.


SEQUENCE LISTING (24 sequences)

SEQ ID NO: 1 - DNA, 60 bp, Synthetic construct
  source 1..60: mol_type = other DNA
atgtacaggatgcaactcctgtcttgcattgcactaagtcttgcacttgtcacaaacagt

SEQ ID NO: 2 - DNA, 399 bp, Synthetic construct
  source 1..399: mol_type = other DNA
ggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 3 - DNA, 459 bp, Synthetic construct
  source 1..459: mol_type = other DNA
atgtacaggatgcaactcctgtcttgcattgcactaagtcttgcacttgtcacaaacagtggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 4 - protein, 20 aa, Synthetic construct
MYRMQLLSCIALSLALVTNS

SEQ ID NO: 5 - protein, 133 aa, Synthetic construct
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 153 aa, Synthetic construct
MYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 370 aa, Synthetic construct
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 8 - protein, 1106 aa, Synthetic construct
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 9 - protein, 140 aa, Synthetic construct
MSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAY

SEQ ID NO: 10 - protein, 174 aa, Synthetic construct
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYD

SEQ ID NO: 11 - DNA, 23 bp, Synthetic construct
  source 1..23: mol_type = other DNA
tgatgccgaggaactattcatct

SEQ ID NO: 12 - DNA, 21 bp, Synthetic construct
  source 1..21: mol_type = other DNA
tttcttctcgtgcagtgtcac

SEQ ID NO: 13 - DNA, 22 bp, Synthetic construct
  source 1..22: mol_type = other DNA
ttgtaccgaagagatgagacca

SEQ ID NO: 14 - DNA, 23 bp, Synthetic construct
  source 1..23: mol_type = other DNA
gctgtatccgtgtattctcctga

SEQ ID NO: 15 - DNA, 21 bp, Synthetic construct
  source 1..21: mol_type = other DNA
tgtgccgctagaggtgaaatt

SEQ ID NO: 16 - DNA, 19 bp, Synthetic construct
  source 1..19: mol_type = other DNA
tggcaaatgctttcgcttt

SEQ ID NO: 17 - DNA, 33 bp, Synthetic construct
  source 1..33: mol_type = other DNA
cggggtacccaaagacctggccaggccccctct

SEQ ID NO: 18 - DNA, 34 bp, Synthetic construct
  source 1..34: mol_type = other DNA
ccgctcgagctggcagcctcaggagctcacacca

SEQ ID NO: 19 - DNA, 34 bp, Synthetic construct
  source 1..34: mol_type = other DNA
ccgctcgagcaaagagattaggaactttatttct

SEQ ID NO: 20 - DNA, 32 bp, Synthetic construct
  source 1..32: mol_type = other DNA
cccaagctttgacgggacaaacaacaggttga

SEQ ID NO: 21 - DNA, 21 bp, Synthetic construct
  source 1..21: mol_type = other DNA
aaatgatctccctgggtgcca

SEQ ID NO: 22 - DNA, 19 bp, Synthetic construct
  source 1..19: mol_type = other DNA
cgcgtgcgtctgttttcaa

SEQ ID NO: 23 - DNA, 21 bp, Synthetic construct
  source 1..21: mol_type = other DNA
tccttagtgtctctcccaggg

SEQ ID NO: 24 - DNA, 22 bp, Synthetic construct
  source 1..22: mol_type = other DNA
aaatttaggtttgtgggccatg